(19) United States
(12) Patent Application Publication
(10) Pub. No.: US 2026/0315391 A1
(43) Pub. Date: Oct. 15, 2026

(54) EXTRACELLULAR VESICLE COMPOSITIONS AND PREPARATION

(71) Applicant: VIRGINIA TECH INTELLECTUAL PROPERTIES, INC., Blacksburg, VA (US)

(72) Inventors: Spencer MARSH, Blacksburg, VA (US); Robert G. GOURDIE, Blacksburg, VA (US); LInda Jane JOURDAN, Blacksburg, VA (US); Alan DOGAN, Blacksburg, VA (US)

(73) Assignee: VIRGINIA TECH INTELLECTUAL PROPERTIES, INC., Blacksburg, VA (US)

(21) Appl. No.: 19/481,842

(22) PCT Filed: May 9, 2024
(86) PCT No.: PCT/US2024/028635

§ 371 Date: Nov. 5, 2025

Related U.S. Application Data
(60) Provisional application No. 63/465,073, filed on May 9, 2023.
(60) Provisional application No. 63/624,637, filed on Jan. 24, 2024.
(60) Provisional application No. 63/640,825, filed on Apr. 30, 2024.

(51) Int. Cl. A61K 9/1276; A61K 9/19; A61K 31/405; A61K 47/26

(52) U.S. Cl. CPC A61K 9/1276; A61K 9/19; A61K 31/405; A61K 47/26

(57) ABSTRACT

In various aspects and embodiments provided are compositions containing extracellular vesicles (including modified extracellular vesicles), methods of making preparations of extracellular vesicles, methods of modifying extracellular vesicles and methods of using modified and/or unmodified extracellular vesicles.

### CROSS-REFERENCE TO RELATED APPLICATIONS

The present application claims the benefit of priority to U.S. Provisional Application Ser. No. 63/465,073, filed on May 9, 2023, and to U.S. Provisional Application Serial No. 63/624,637, filed on Jan. 24, 2024, and to U.S. Provisional Application Ser. No. 63/640,825, filed on Apr. 30, 2024, all of which are incorporated by reference in their entirety.

### STATEMENT REGARDING FEDERALLY SPONSORED RESEARCH

This invention was made with government support under Grant No. HL161237, HL141855, and HL056728 awarded by the National Institutes of Health and STTR #2203330 by the NSF. The government has certain rights in the invention.

### FIELD OF THE DISCLOSURE

The present disclosure relates generally to extracellular vesicles (for example milk-derived vesicles, or exosomes) as well as methods of making, modifying and using such vesicles.

### BACKGROUND

PCT Application Publication Number WO2022182782 discloses “methods of isolating exosomes from a biological fluid, such as those containing caseins.” PCT Application Publication Number WO2020010161 discloses “milk vesicles as drug delivery vehicles, compositions comprising a therapeutic agent encapsulated within or otherwise associated with the milk vesicles, methods of producing such milk vesicles and compositions thereof, as well as methods of delivering such milk vesicles and compositions to a specific patient tissue or organ.”

### SUMMARY

The instant disclosure is based, at least in part, on compositions containing extracellular vesicles (including modified extracellular vesicles), methods of making preparations of isolated extracellular vesicles, methods of modifying extracellular vesicles and methods of using modified and/or unmodified extracellular vesicles.

The terms “extracellular vesicle” or “EV” or “vesicle” as used herein are used interchangeably and refer to a small, lipid-bilayer membrane bound structure that exists outside of a cell. In many embodiments, extracellular vesicles are released from cells into the extracellular space. Extracellular vesicles have a lipid bilayer and are generally about 30 to 1,000 nm in diameter. In addition to the lipid bilayer and internal aqueous lumen, extracellular vesicles have additional components such as tetraspanin proteins and IgG molecules; accordingly, detection of these components in isolated extracellular vesicles can be used to distinguish other (such as synthetic) particles having lipid bilayers. Extracellular vesicles often contain natural bioactive agents in their interior and/or in the outer bilayer membrane. Further, as discussed in more detail below, extracellular vesicles can be loaded with “cargo” such as therapeutic molecules and the cargo-loaded vesicles can enhance delivery of the cargo when administered to a subject; e.g., cargo-loaded vesicles in certain conditions can be used to effectively orally administer molecules that are otherwise not bioavailable or poorly bioavailable when administered orally.

Milk has been found to be a rich source of extracellular vesicles, and recent studies have shown that isolated extracellular vesicle preparations can be made from milk can be loaded with “cargo” (such as therapeutic molecules) and can be used to successfully administer the cargo to subjects (see for example WO2022182782).

Certain aspects and embodiments of the instant disclosure are based on discoveries that isolated extracellular vesicles that are modified to have ATP and/or one or more ATP analogues can increase the cellular uptake of the modified extracellular vesicles when administered to a subject, thereby increasing the effectiveness of administration of a cargo if present in the vesicles. The term ATP means adenosine triphosphate. As used herein, the term “ATP-analogue-vesicle” refers to an extracellular vesicle that has been modified to include ATP and/or one or more ATP analogues. In certain embodiments, the ATP-analogue-vesicles of the disclosure may be particularly useful for administration of a cargo via a route other than oral administration, such as parenteral administration, intranasal administration, IV administration, injection, subcutaneous injection or topical administration.

Accordingly, in one aspect provided is a composition comprising isolated ATP-analogue vesicles wherein said vesicles comprise between 0.01 and 1,000 μM ATP or an ATP analogue. In a similar aspect, provided is a composition comprising isolated milk ATP-analogue vesicles wherein said vesicles comprise between 0.01 and 1,000 μM ATP or an ATP analogue. In yet another aspect a composition is provided wherein the composition includes isolated bovine milk ATP-analogue vesicles, wherein said vesicles comprise between 0.01 and 1,000 μM ATP or an ATP analogue. In some embodiments the ATP-analogue vesicles include ATP or an ATP analogue in a concentration between 0.01 and 1,000 μM; or 0.1 and 100 μM; or 0.25 and 175 μM; 0.5 and 50 μM; or 0.75 and 25 μM; or 0.8 and 20 μM; or 0.85 and 15 μM; or 0.9 and 10 μM; or 0.1 and 5 μM; or 0.5 and 5 μM; or 0.1 and 2 μM; or 0.5 and 1.5 μM; or 0.75 and 1.25 μM. In some embodiments the ATP-analogue vesicles include ATP or an ATP analogue in a concentration of about 0.1 μM; 0.25 μM; 0.5 μM; 0.75 μM; 0.9 μM; 1 μM; 1.1 μM; 1.25 μM; 1.5 μM; 2 μM; 5 μM; 10 μM; 50 μM; or 100 μM. Herein when an agent (such as ATP) in vesicles is described in terms of molarity (for example as μM), the vesicles are exposed to a solution of the agent at the indicated molarity until an equilibrium is achieved (for example using methods described herein) such that the molarity within each vesicle is congruent with the molar concentration of the solution.

In another aspect provided is a composition comprising isolated ATP-analogue vesicles wherein said vesicles comprise between 1 ng and 1,000 μg ATP or an ATP analogue per mg of vesicle protein. In a similar aspect, provided is a composition comprising isolated milk ATP-analogue vesicles wherein said vesicles comprise between 1 ng and 1,000 μg ATP or an ATP analogue per mg of milk vesicle protein. In yet another aspect a composition is provided wherein the composition includes isolated bovine milk ATP-analogue vesicles, wherein said vesicles comprise between 1 ng and 1,000 μg ATP or an ATP analogue per mg of bovine milk ATP-analogue vesicles. In some embodiments the ATP-analogue vesicles include ATP or an ATP analogue in a concentration between 1 ng and 1,000 μg per mg vesicle protein; or 10 ng and 100 μg per mg vesicle protein; or 100 ng and 10 μg per mg vesicle protein; or 200 ng and 1 μg per mg vesicle protein; or 300 ng and 750 ng per mg vesicle protein; or 400 ng and 650 ng per mg vesicle protein; or 450 ng and 600 ng per mg vesicle protein; or 500 ng and 550 ng per mg vesicle protein. In some embodiments, the ATP-analogue vesicles include ATP or an ATP analogue in a concentration of about 1 ng per mg vesicle protein; 10 ng per mg vesicle protein; 100 ng per mg vesicle protein; 200 ng per mg vesicle protein; 300 ng per mg vesicle protein; 400 ng per mg vesicle protein; 450 ng per mg vesicle protein; 500 ng per mg vesicle protein; 550 ng per mg vesicle protein; 600 ng per mg vesicle protein; 650 ng per mg vesicle protein; 750 ng per mg vesicle protein; 1 μg per mg vesicle protein; 10 μg per mg vesicle protein; 100 μg per mg vesicle protein; or 1,000 μg per mg vesicle protein.

In a related aspect, a method is provided that includes obtaining a composition that comprises isolated vesicles (such as, for example any isolated vesicle composition as described herein), contacting the vesicles with a solution comprising ATP and/or an ATP analogue to obtain a composition comprising ATP-analogue-vesicles. As used herein, the term “isolated” or the like refers to an agent (for example a vesicle), which has been separated from its original environment, i.e., the environment of the isolated agent (for example a vesicle) is substantially free of at least one component as found in the environment in which the “un-isolated” reference agents (for example a vesicles) exist. The term includes an agent (for example a vesicle) that is removed from some or all components as it is found in its natural environment, for example, isolated from a tissue, biopsy body fluid (such as milk) sample. The term also includes an agent (for example a vesicle) that is removed from at least one, some or all components as the agent (for example a vesicle) is found in non-naturally occurring environments, for example, isolated from a cell culture or cell suspension. Therefore, an isolated agent (for example a vesicle) is partly or completely separated from at least one component, including other substances, cells or cell populations, as it is found in nature or as it is grown, stored or subsisted in non-naturally occurring environments. In some embodiments, an isolated vesicle of the disclosure is an isolated extracellular vesicle that has been isolated from milk, for example a vesicle that has been isolated from milk using one or more of the methods described herein and/or described in PCT WO2022182782. Vesicles isolated from milk are referred to herein as “milk vesicles.”

In certain aspects and embodiments, the vesicles herein are provided in a liquid solution. As used herein, vesicles in a “liquid solution” means that the vesicles are present in a liquid, for example the liquid may be one or more solvents such as water, vegetable oils, terpenes, aldehydes, ketones, diacetone alcohol, ethylene glycol ethers, methanol, ethanol, isopropanol. In certain embodiments the liquid is an aqueous liquid. In certain embodiments the liquid is a physiological solution having electrolytes and or buffering agents. In certain embodiments the liquid may also include one or more solutes including sodium chloride, potassium chloride and other salts, magnesium, calcium, disaccharides, amino acids, nucleic acids, and further solute solutions known to those skilled in the art.

In certain aspects and embodiments, the extracellular vesicle composition is provided in a dried, or lyophilized state. The term “dried” and “lyophilized” as used herein means a product such as a cake or powder from which water has been removed through freeze drying or sublimation, in which the final product has a lower water concentration than the original liquid starting material. In certain embodiments, other forms of drying or “lyophilizing” as stated herein may include spray drying, drug drying, crystallization, extrusion and other forms of drying known to those skilled in the art.

In certain aspects and embodiments, the dried product has a moisture content (defined by [mass of water/mass of sample)*100]) of less than about10%; or less than about 9%; or less than about 8%; or less than about 7%; or less than about 6%; or less than about 5%; or less than about 4%; or less than about 3%; or less than about 2%; or less than about 1%; or less than about 0.5%; or less than about 0.1% moisture by weight.

The term “analogue” as used herein means a chemical compound that is similar to another chemical compound in structure and/or function, but differs structurally from that compound. Such difference may be by one single element or group, or more than one group (e.g., 1, 2, 3, 4 or more groups), provided that it retains the same chemical scaffold and preferably function as the parental chemical. Such modifications are routine to persons skilled in the art, and include, for example, additional or substituted chemical moieties. In some embodiments, an analogue as used herein may have a chemical structure that is different, or very different, from the parental compound but have similarities in a relevant function or biological activity. In some embodiments, an analogue as used herein may have a chemical structure that is different, or very different, from the parental compound but have at least one relevant function or biological activity increased, decreased, or deleted.

As used herein, the term “ATP analogue” refers to an analogue of ATP. In certain embodiments, an ATP analogue of the disclosure is a non-hydrolyzable analogue of ATP. In some embodiments an ATP analogue of the disclosure is one or more selected from the group consisting of α,62 -methylene-ATP (α,62 mATP); β,γmethylene-ATP (β, γmATP); 2-thio-ATP (2-SH-ATP); 2-methylthio-ATP (2-MeS-ATP); 2′,3′-O-2,4,6,-trinitrophenyl-ATP (TNP-ATP); 2′,3′-9-(4-benzoyl)-ATP (BzATP); an N-alkyl-2 ATP; adenosine 5′-(β,γimido)triphosphate (AMP-PNP); ATP-MgCl2; 5-aminoimidazole-4-carboxamide ribonucleotide, disoproxil fumarate, ribavirin, azidothymidine, fludarabine, efavirenz and oxidized ATP (oATP), Adenosine, ADP, AMP, and other nucleotides (e.g. GTP, CTP, UTP, TTP).

As used herein, the term “Tryptophan analogue” refers to tryptophan itself or an analogue of tryptophan. In certain embodiments, a tryptophan analogue of the disclosure is Tryptophan. In some embodiments a tryptophan analogue of the disclosure is one or more selected from the group consisting of L-Tryptophan, D-Tryptophan, Tryptophan dimers, Tryptophan trimers, Tryptophan peptides, Histidine, L-Histidine, D-Histidine, Histidine dimers, Histidine trimers, Histidine peptides, Tyrosine, L-Tyrosine, D-Tyrosine, Tyrosine dimers, Tyrosine trimers, Tyrosine peptides, Arginine, L-Arginine, D-Arginine, Arginine dimers, Arginine trimers, Arginine peptides, Cysteine, L-Cysteine, D-Cysteine, Cysteine dimers, Cysteine trimers, Cysteine peptides, Lysine, L-Lysine, D-Lysine, Lysine dimers, Lysine trimers, Lysine Peptides, Phenylalanine, L-Phenylalanine, D-Phenylalanine, Phenylalanine dimers, Phenylalanine trimers, Phenylalanine peptides, D-Carnitine, L-Carnitine, Carnitine dimers, Carnitine trimers, Carnitine peptides, Acetyl L-Carnitine, L-Carnitine L-tartrate, Propionyl-L-carnitine, D-Taurine, L-Taurine, Taurine dimers, Taurine trimers, Taurine peptides.

Other aspects and embodiments of the disclosure are based on discoveries that certain molecules such as tryptophan and/or tryptophan analogues can be used to disrupt associations of extracellular vesicles with casein and other milk protein molecules that can aggregate and complicate the isolation of extracellular vesicles from milk; and associated methods of isolating extracellular vesicles from milk wherein the methods involve contacting extracellular vesicle containing milk with tryptophan and/or tryptophan analogues.

Accordingly, in one aspect provided is a method of isolating extracellular vesicles from a milk sample, wherein the method involves contacting (or incubating) the milk sample with tryptophan and/or one or more tryptophan analogues and subsequently separating extracellular vesicles from one or more milk components (such as for example casein-containing aggregates). In some embodiments the method includes contacting (incubating) the vesicle-containing sample with tryptophan and/or or more tryptophan analogues at a concentration between 10 μM and 1 mM. In some embodiments the method includes contacting (incubating) the vesicle-containing sample with tryptophan and/or or more tryptophan analogues at a temperature between 4 and 50 degrees Celsius for at least 10 minutes. In some embodiments the method includes contacting (incubating) the vesicle containing sample with tryptophan and/or 1 or more tryptophan analogues at a concentration between 10 μM and 1 mM at a temperature between 10 and 50 degrees Celsius for at least 10 minutes. In some embodiments the method includes contacting (incubating) the vesicle containing sample with tryptophan and/or 1 or more tryptophan analogues at a concentration between 10 μM and 1 mM at a temperature between 10 and 50 degrees Celsius for between 10 and 60 minutes.

In various embodiments an extracellular vesicle of the compositions and methods of the disclosure has a diameter between (or in the case of a composition that includes a plurality of vesicles, the average (mean) diameter of the particles is between), 1 and 2,000 nm; or 10 and 1,500 nm; or 20 and 1,000 nm; or 1 and 1,000 nm; or 1 and 500 nm; or 1 and 300 nm; or 1 and 200 nm; or 1 and 185 nm, or 1 and 175 nm; or 1 and 170 nm; or 1 and 165 nm; or 1 and 160 nm; or 1 and 155 nm; or 1 and 150 nm; or 1 and 145 nm; or 1 and 140 nm; or 1 and 135 nm; or 1 and 130 nm; or 10 and 1,000 nm; or 10 and 500 nm; or 10 and 300 nm; or 10 and 200 nm; or 10 and 185 nm, or 10 and 175 nm; or 10 and 170 nm; or 10 and 165 nm; or 10 and 160 nm; or 10 and 155 nm; or 10 and 150 nm; or 10 and 145 nm; or 10 and 140 nm; or 10 and 135 nm; or 10 and 130 nm; or 15 and 1,000 nm; or 15 and 500 nm; or 15 and 300 nm; or 15 and 200 nm; or 15 and 185 nm, or 15 and 175 nm; or 15 and 170 nm; or 15 and 165 nm; or 15 and 160 nm; or 15 and 155 nm; or 15 and 150 nm; or 15 and 145 nm; or 15 and 140 nm; or 15 and 135 nm; or 15 and 130 nm; or 20 and 1,000 nm; or 20 and 500 nm; or 20 and 300 nm; or 20 and 200 nm; or 20 and 185 nm, or 20 and 175 nm; or 20 and 170 nm; or 20 and 165 nm; or 20 and 160 nm; or 20 and 155 nm; or 20 and 150 nm; or 20 and 145 nm; or 20 and 140 nm; or 20 and 135 nm; or 20 and 130 nm; or 25 and 1,000 nm; or 25 and 500 nm; or 25 and 300 nm; or 25 and 200 nm; or 25 and 185 nm, or 25 and 175 nm; or 25 and 170 nm; or 25 and 165 nm; or 25 and 160 nm; or 25 and 155 nm; or 25 and 150 nm; or 25 and 145 nm; or 25 and 140 nm; or 25 and 135 nm; or 25 and 130 nm; or 30 and 1,000 nm; or 30 and 500 nm; or 30 and 300 nm; or 30 and 200 nm; or 30 and 185 nm, or 30 and 175 nm; or 30 and 170 nm; or 30 and 165 nm; or 30 and 160 nm; or 30 and 155 nm; or 30 and 150 nm; or 30 and 145 nm; or 30 and 140 nm; or 30 and 135 nm; or 30 and 130 nm; or 35 and 1,000 nm; or 35 and 500 nm; or 35 and 300 nm; or 35 and 200 nm; or 35 and 185 nm, or 35 and 175 nm; or 35 and 170 nm; or 35 and 165 nm; or 35 and 160 nm; or 35 and 155 nm; or 35 and 150 nm; or 35 and 145 nm; or 35 and 140 nm; or 35 and 135 nm; or 35 and 130 nm; or 40 and 1,000 nm; or 40 and 500 nm; or 40 and 300 nm; or 40 and 200 nm; or 40 and 185 nm, or 40 and 175 nm; or 40 and 170 nm; or 40 and 165 nm; or 40 and 160 nm; or 40 and 155 nm; or 40 and 150 nm; or 40 and 145 nm; or 40 and 140 nm; or 40 and 135 nm; or 40 and 130 nm; or 45 and 1,000 nm; or 45 and 500 nm; or 45 and 300 nm; or 45 and 200 nm; or 45 and 185 nm, or 45 and 175 nm; or 45 and 170 nm; or 45 and 165 nm; or 45 and 160 nm; or 45 and 155 nm; or 45 and 150 nm; or 45 and 145 nm; or 45 and 140 nm; or 45 and 135 nm; or 45 and 130 nm; or 50 and 1,000 nm; or 50 and 500 nm; or 50 and 300 nm; or 50 and 200 nm; or 50 and 185 nm, or 50 and 175 nm; or 50 and 170 nm; or 50 and 165 nm; or 50 and 160 nm; or 50 and 155 nm; or 50 and 150 nm; or 50 and 145 nm; or 50 and 140 nm; or 50 and 135 nm; or 50 and 130 nm; or 55 and 1,000 nm; or 55 and 500 nm; or 55 and 300 nm; or 55 and 200 nm; or 55 and 185 nm, or 55 and 175 nm; or 55 and 170 nm; or 55 and 165 nm; or 55 and 160 nm; or 55 and 155 nm; or 55 and 150 nm; or 55 and 145 nm; or 55 and 140 nm; or 55 and 135 nm; or 55 and 130 nm; or 60 and 1,000 nm; or 60 and 500 nm; or 60 and 300 nm; or 60 and 200 nm; or 60 and 185 nm, or 60 and 175 nm; or 60 and 170 nm; or 60 and 165 nm; or 60 and 160 nm; or 60 and 155 nm; or 60 and 150 nm; or 60 and 145 nm; or 60 and 140 nm; or 60 and 135 nm; or 60 and 130 nm; or 65 and 1,000 nm; or 65 and 500 nm; or 65 and 300 nm; or 65 and 200 nm; or 65 and 185 nm, or 65 and 175 nm; or 65 and 170 nm; or 65 and 165 nm; or 65 and 160 nm; or 65 and 155 nm; or 65 and 150 nm; or 65 and 145 nm; or 65 and 140 nm; or 65 and 135 nm; or 65 and 130 nm; or 70 and 1,000 nm; or 70 and 500 nm; or 70 and 300 nm; or 70 and 200 nm; or 70 and 185 nm, or 70 and 175 nm; or 70 and 170 nm; or 70 and 165 nm; or 70 and 160 nm; or 70 and 155 nm; or 70 and 150 nm; or 70 and 145 nm; or 70 and 140 nm; or 70 and 135 nm; or 70 and 130 nm; or 75 and 1,000 nm; or 75 and 500 nm; or 75 and 300 nm; or 75 and 200 nm; or 75 and 185 nm, or 75 and 175 nm; or 75 and 170 nm; or 75 and 165 nm; or 75 and 160 nm; or 75 and 155 nm; or 75 and 150 nm; or 75 and 145 nm; or 75 and 140 nm; or 75 and 135 nm; or 75 and 130 nm; or 90 and 1,000 nm; or 90 and 500 nm; or 90 and 300 nm; or 90 and 200 nm; or 90 and 185 nm, or 90 and 175 nm; or 90 and 170 nm; or 90 and 165 nm; or 90 and 160 nm; or 90 and 155 nm; or 90 and 150 nm; or 90 and 145 nm; or 90 and 140 nm; or 90 and 135 nm; or 90 and 130 nm; or 95 and 1,000 nm; or 95 and 500 nm; or 95 and 300 nm; or 95 and 200 nm; or 95 and 185 nm, or 95 and 175 nm; or 95 and 170 nm; or 95 and 165 nm; or 95 and 160 nm; or 95 and 155 nm; or 95 and 150 nm; or 95 and 145 nm; or 95 and 140 nm; or 95 and 135 nm; or 95 and 130 nm; or 100 and 1,000 nm; or 100 and 500 nm; or 100 and 300 nm; or 100 and 200 nm; or 100 and 185 nm, or 100 and 175 nm; or 100 and 170 nm; or 100 and 165 nm; or 100 and 150 nm; or 100 and 155 nm; or 100 and 150 nm; or 100 and 145 nm; or 100 and 100 nm; or 100 and 135 nm; or 100 and 130 nm; or 105 and 1,000 nm; or 105 and 500 nm; or 105 and 300 nm; or 105 and 200 nm; or 105 and 185 nm, or 105 and 175 nm; or 105 and 170 nm; or 105 and 165 nm; or 105 and 160 nm; or 105 and 155 nm; or 105 and 150 nm; or 105 and 145 nm; or 105 and 140 nm; or 105 and 135 nm; or 105 and 130 nm; or 110 and 1,000 nm; or 110 and 500 nm; or 110 and 300 nm; or 110 and 200 nm; or 110 and 185 nm, or 110 and 175 nm; or 110 and 170 nm; or 110 and 165 nm; or 110 and 150 nm; or 110 and 155 nm; or 110 and 150 nm; or 110 and 145 nm; or 110 and 100 nm; or 110 and 135 nm; or 110 and 130 nm; or 115 and 1,000 nm; or 115 and 500 nm; or 115 and 300 nm; or 115 and 200 nm; or 115 and 185 nm, or 115 and 175 nm; or 115 and 170 nm; or 115 and 165 nm; or 115 and 160 nm; or 115 and 155 nm; or 115 and 150 nm; or 115 and 145 nm; or 115 and 140 nm; or 115 and 135 nm; or 115 and 130 nm; or 120 and 1,000 nm; or 120 and 500 nm; or 120 and 300 nm; or 120 and 200 nm; or 120 and 185 nm, or 120 and 175 nm; or 120 and 170 nm; or 120 and 165 nm; or 120 and 150 nm; or 120 and 155 nm; or 120 and 150 nm; or 120 and 145 nm; or 120 and 100 nm; or 120 and 135 nm; or 120 and 130 nm.

In some embodiments, it may be preferable to have all, or most of the extracellular vesicle particles in a composition to be less than about 600 nm, or in some embodiments, less than about 500 nm, in size. For example, extracellular vesicles used to deliver therapeutic cargo can, in some situations, attract an immune response in a subject when there are particle sizes over 500 or 600 nm. In certain embodiments, extracellular vesicles with particle sizes over about 600 nm, or over about 500 nm, or over about 250 nm can be removed using filtration, for example using a 0.2 μm (or 0.22 μm) filter or the like. Accordingly, in some embodiments at least 75%, or 80%; or 90%, or 95%, or 98%, or 99% of the extracellular vesicles in an isolated vesicle composition of the disclosure have a particle size of less than about 600 nm; or less than about 500 nm; or less than about 250 nm.

In some embodiments, it may be preferable to have all, or most of the extracellular vesicle particles in a composition to be more than about 20 nm in size. For example, extracellular vesicles used to deliver therapeutic cargo can, in some situations, be less effective when there is a substantial amount of particles with a size less than about 20 nm in size. In certain embodiments, extracellular vesicles with particle sizes below about 20 nm can be removed using filtration, for example using a 500 kDa filter or the like. Accordingly, in some embodiments at least 75%, or 80%; or 90%, or 95%, or 98%, or 99% of the vesicles in an isolated extracellular vesicle composition of the disclosure have a particle size greater than about 20 nm.

In some embodiments, it may be preferable to have all, or most of the extracellular vesicle particles in a composition to be more than about 20 nm in size and also having vesicles to be less than about 600 nm (or in some embodiments, less than about 500 nm) in size. Accordingly, in some embodiments at least 75%, or 80%; or 90%, or 95%, or 98%, or 99% of the extracellular vesicles in an isolated vesicle composition of the disclosure have a particle size of less than about 600 nm; or less than about 500 nm; or less than about 250 nm; and at least 75%, or 80%; or 90%, or 95%, or 98%, or 99% of the vesicles in an isolated vesicle composition of the disclosure have a particle size greater than about 20 nm.

In some embodiments, extracellular vesicles of the disclosure (such as isolated milk extracellular vesicles; and/or ATP-analogue-vesicles) include one or more cargo molecules. As used herein the term “cargo” means a molecule (for example a biological molecule) that is added to the vesicles, for example a molecule that is intended to be administered to a subject using the vesicles as a delivery vehicle. In some embodiments a cargo molecule is a peptide, protein, nucleic acid, polysaccharide, a small molecule, or the like. In many embodiments, the cargo molecule is not bioavailable, or has poor bioavailability when administered by a particular route, but has increased or improved bioavailability when administered through a vesicle composition of the present disclosure. In some embodiments, a cargo molecule has a molecular weight that is greater than 20 Daltons and less than 500,000 Daltons, between 2 and 1000 amino acids, or between 1 picometer and 200 nanometers in size. In some embodiments the cargo is a molecule that is not naturally present in, or endogenous to, the vesicles in their natural source. In some embodiments, the cargo molecule may be endogenous to the milk vesicle in its natural source but is added exogenously, for example to increase the amount of the molecule in the vesicle. Non limiting exemplary molecules that may serve as cargo include an antibody, a hormone, a growth factor, an enzyme, a cytokine, a chemokine, a toxin, an antitoxin, a blood coagulation factor, an interfering RNA (iRNA), a micro RNA (miRNA), an antisense RNA, a messenger RNA (mRNA), a non-coding RNA, a single-stranded DNA (ssDNA), a double-stranded DNA (dsDNA), long non-coding RNA, and an iRNA (such as, for example, siRNA or shRNA). Any of the nucleotide molecules disclosed herein, or a fragment thereof, may comprise a naturally-occurring nucleotide sequence. Alternatively, the nucleotide molecules can be synthetic (non-naturally occurring).

### DETAILED DESCRIPTION

### Vesicle Sizes, Types and Determination Thereof

As used herein, the terms “size” and “diameter” are used interchangeably. In embodiments that involve a composition that includes a plurality of vesicles (such as milk vesicles, exosomes, milk exosomes, milk extracellular vesicles, etc. ,), the average (mean) size or diameter of the vesicles in the composition may be any of the sizes indicated herein for a single vesicle (for example, in the case a size range is indicated, the average size of the particles fall inside the specified range. Vesicles of the disclosure can be of any size specified herein. In certain embodiments, and where specifically indicated, when size (diameter) is represented as a range and in the case of a composition including a plurality of vesicles, the average size of the particles falls within the stated range and a specific percentage of the particles e.g., more than 60%; or more than 65%; or more than 70%; or more than 75%; or more than 80%; or more than 85%; or more than 90%; or more than 95% of the vesicle particles fall within the indicated range.

Particle size can be determined by nanoparticle tracking analysis (NTA) or dynamic light scattering (DLS), or microfluidic resistive pulse sensing.

As used herein, the term “exosome” refers to a subset of extracellular vesicles that are generally characterized by having a specific size but can also be characterized by additional unique features, such as relatively increased amounts of protein markers such as CD81, CD9 and syntenin. Exosomes are characterized by being of a size under 200 nanometers, containing tetraspanin proteins including CD81, CD9 and CD63, luminal proteins including syntenin and TSG-101, is comprised of a lipid bilayer membrane and stably transfer cargoes between cells. In some embodiments an exosome (such as a milk exosome) of the disclosure has a diameter between (or in the case of a composition that includes a plurality of vesicles, the average (mean) diameter of the particles is between) 10 and 200 nm. In some embodiments an exosome (such as a milk exosome) of the disclosure has a diameter between (or in the case of a composition that includes a plurality of vesicles, the average (mean) diameter of the particles is between) 10 and 200 nm; or 10 and 185 nm, or 10 and 175 nm; or 10 and 170 nm; or 10 and 165 nm; or 10 and 160 nm; or 10 and 155 nm; or 10 and 150 nm; or 10 and 145 nm; or 10 and 140 nm; or 10 and 135 nm; or 10 and 130 nm; or 15 and 200 nm; or 15 and 185 nm, or 15 and 175 nm; or 15 and 170 nm; or 15 and 165 nm; or 15 and 160 nm; or 15 and 155 nm; or 15 and 150 nm; or 15 and 145 nm; or 15 and 140 nm; or 15 and 135 nm; or 15 and 130 nm; or 20 and 200 nm; or 20 and 185 nm, or 20 and 175 nm; or 20 and 170 nm; or 20 and 165 nm; or 20 and 160 nm; or 20 and 155 nm; or 20 and 150 nm; or 20 and 145 nm; or 20 and 140 nm; or 20 and 135 nm; or 20 and 130 nm; or 25 and 200 nm; or 25 and 185 nm, or 25 and 175 nm; or 25 and 170 nm; or 25 and 165 nm; or 25 and 160 nm; or 25 and 155 nm; or 25 and 150 nm; or 25 and 145 nm; or 25 and 140 nm; or 25 and 135 nm; or 25 and 130 nm; or 30 and 200 nm; or 30 and 185 nm, or 30 and 175 nm; or 30 and 170 nm; or 30 and 165 nm; or 30 and 160 nm; or 30 and 155 nm; or 30 and 150 nm; or 30 and 145 nm; or 30 and 140 nm; or 30 and 135 nm; or 30 and 130 nm; or 35 and 200 nm; or 35 and 185 nm, or 35 and 175 nm; or 35 and 170 nm; or 35 and 165 nm; or 35 and 160 nm; or 35 and 155 nm; or 35 and 150 nm; or 35 and 145 nm; or 35 and 140 nm; or 35 and 135 nm; or 35 and 130 nm; or 40 and 200 nm; or 40 and 185 nm, or 40 and 175 nm; or 40 and 170 nm; or 40 and 165 nm; or 40 and 160 nm; or 40 and 155 nm; or 40 and 150 nm; or 40 and 145 nm; or 40 and 140 nm; or 40 and 135 nm; or 40 and 130 nm; or 45 and 200 nm; or 45 and 185 nm, or 45 and 175 nm; or 45 and 170 nm; or 45 and 165 nm; or 45 and 160 nm; or 45 and 155 nm; or 45 and 150 nm; or 45 and 145 nm; or 45 and 140 nm; or 45 and 135 nm; or 45 and 130 nm; or 50 and 200 nm; or 50 and 185 nm, or 50 and 175 nm; or 50 and 170 nm; or 50 and 165 nm; or 50 and 160 nm; or 50 and 155 nm; or 50 and 150 nm; or 50 and 145 nm; or 50 and 140 nm; or 50 and 135 nm; or 50 and 130 nm; or 55 and 200 nm; or 55 and 185 nm, or 55 and 175 nm; or 55 and 170 nm; or 55 and 165 nm; or 55 and 160 nm; or 55 and 155 nm; or 55 and 150 nm; or 55 and 145 nm; or 55 and 140 nm; or 55 and 135 nm; or 55 and 130 nm; or 60 and 200 nm; or 60 and 185 nm, or 60 and 175 nm; or 60 and 170 nm; or 60 and 165 nm; or 60 and 160 nm; or 60 and 155 nm; or 60 and 150 nm; or 60 and 145 nm; or 60 and 140 nm; or 60 and 135 nm; or 60 and 130 nm; or 65 and 200 nm; or 65 and 185 nm, or 65 and 175 nm; or 65 and 170 nm; or 65 and 165 nm; or 65 and 160 nm; or 65 and 155 nm; or 65 and 150 nm; or 65 and 145 nm; or 65 and 140 nm; or 65 and 135 nm; or 65 and 130 nm; or 70 and 200 nm; or 70 and 185 nm, or 70 and 175 nm; or 70 and 170 nm; or 70 and 165 nm; or 70 and 160 nm; or 70 and 155 nm; or 70 and 150 nm; or 70 and 145 nm; or 70 and 140 nm; or 70 and 135 nm; or 70 and 130 nm; or 75 and 200 nm; or 75 and 185 nm, or 75 and 175 nm; or 75 and 170 nm; or 75 and 165 nm; or 75 and 160 nm; or 75 and 155 nm; or 75 and 150 nm; or 75 and 145 nm; or 75 and 140 nm; or 75 and 135 nm; or 75 and 130 nm; or 80 and 200 nm; or 80 and 185 nm, or 80 and 175 nm; or 80 and 170 nm; or 80 and 165 nm; or 80 and 160 nm; or 80 and 155 nm; or 80 and 150 nm; or 80 and 145 nm; or 80 and 140 nm; or 80 and 135 nm; or 80 and 130 nm; or 85 and 200 nm; or 85 and 185 nm, or 85 and 175 nm; or 85 and 170 nm; or 85 and 165 nm; or 85 and 160 nm; or 85 and 155 nm; or 85 and 150 nm; or 85 and 145 nm; or 85 and 140 nm; or 85 and 135 nm; or 85 and 130 nm; or 90 and 200 nm; or 90 and 185 nm, or 90 and 175 nm; or 90 and 170 nm; or 90 and 165 nm; or 90 and 160 nm; or 90 and 155 nm; or 90 and 150 nm; or 90 and 145 nm; or 90 and 140 nm; or 90 and 135 nm; or 90 and 130 nm; or 95 and 200 nm; or 95 and 185 nm, or 95 and 175 nm; or 95 and 170 nm; or 95 and 165 nm; or 95 and 160 nm; or 95 and 155 nm; or 95 and 150 nm; or 95 and 145 nm; or 95 and 140 nm; or 95 and 135 nm; or 95 and 130 nm; or 100 and 200 nm; or 100 and 185 nm, or 100 and 175 nm; or 100 and 170 nm; or 100 and 165 nm; or 100 and 150 nm; or 100 and 155 nm; or 100 and 150 nm; or 100 and 145 nm; or 100 and 100 nm; or 100 and 135 nm; or 100 and 130 nm; or 105 and 200 nm; or 105 and 185 nm, or 105 and 175 nm; or 105 and 170 nm; or 105 and 165 nm; or 105 and 160 nm; or 105 and 155 nm; or 105 and 150 nm; or 105 and 145 nm; or 105 and 140 nm; or 105 and 135 nm; or 105 and 130 nm; or 110 and 200 nm; or 110 and 185 nm, or 110 and 175 nm; or 110 and 170 nm; or 110 and 165 nm; or 110 and 150 nm; or 110 and 155 nm; or 110 and 150 nm; or 110 and 145 nm; or 110 and 100 nm; or 110 and 135 nm; or 110 and 130 nm; or 115 and 200 nm; or 115 and 185 nm, or 115 and 175 nm; or 115 and 170 nm; or 115 and 165 nm; or 115 and 160 nm; or 115 and 155 nm; or 115 and 150 nm; or 115 and 145 nm; or 115 and 140 nm; or 115 and 135 nm; or 115 and 130 nm; or 120 and 200 nm; or 120 and 185 nm, or 120 and 175 nm; or 120 and 170 nm; or 120 and 165 nm; or 120 and 150 nm; or 120 and 155 nm; or 120 and 150 nm; or 120 and 145 nm; or 120 and 140 nm; or 120 and 135 nm; or 120 and 130 nm.

### Vesicle Cargo Loading

In certain embodiments, the method further includes loading vesicles of compositions and methods described herein with one or more cargos. The vesicles can be loaded by any suitable method. Exemplary methods of loading the vesicles, such as those prepared by a method described herein, are any of those set forth in International Patent Application Publication W02020/028439, particularly at pages 83-87.

Milk vesicles can be loaded with any suitable or desired cargo(s). In some embodiments, the cargo(s) are therapeutic compounds or molecules. Exemplary cargos include, but are not limited to, DNA, RNA, amino acids, peptides, polypeptides, antibodies, aptamers, ribozymes, hormones, immunomodulators, antipyretics, anxiolytics, antipsychotics, analgesics, antispasmodics, anti-inflammatoirenti-histamines, anti-infectives, radiation sensitizers, chemotherapeutics, imaging agents, immunogens, anti-cancer drugs, any combinations thereof, and/or the like.

In various embodiments, the cargo may be a peptide, such as an ACT-11 peptide. In certain example embodiments, the cargo may be an ACT-11-minus I peptide. Other peptide cargos include those set forth in International Patent Application Publication W02020/028439, particularly at pages 67-82 and 85 and 106-111. In some embodiments, the cargo compound is esterified, such as described in International Patent Application Publication W02020/028439 at page 81-86. In some embodiments, the cargo compound has multiple esterifications, such as described in International Patent Application Publication W02020/028439 at page 81-86.

In certain example embodiments, the exogenous cargo is a biologic molecule. In certain example embodiments, the exogenous cargo is a lipid, polypeptide, peptide, a nucleic acid or a cellular metabolite. In certain example embodiments, the exogenous cargo is a Cx43 carboxyl terminal peptide, ACT1 peptide, ACT11 peptide, an ACT minus I peptide, a selectide, or any combination thereof. In certain example embodiments, the exogenous cargo is any of those cargos set forth in WO 2020/028439, an ACT 1 or ACT 11 peptide, an ACT minus peptide (see e.g., ACT minus peptides set forth in WO 2022/076932), Gap19, JM2 (see e.g., United States Patent No.: U.S. Pat. No. 9,345,744B 2 ), a selectide (see e.g., WO 2022/076932), or any combination thereof. In certain example embodiments, the exogenous cargo is a peptide, including but not limited to an ACT-11 peptide. In certain example embodiments, the exogenous cargo is a peptide, including but not limited to an ACT-11-minus I peptide. Other peptide cargos include those set forth in International Patent Application Publication WO 2020/028439, particularly at pages 67-82 and 85 and 106-111. In some embodiments, the cargo compound is esterified, such as described in International Patent Application Publication WO 2020/028439 at page 81-86. In some embodiments, the cargo compound has multiple esterifications, such as described in International Patent Application Publication WO 2020/028439 at page 81-86. Other exemplary peptidic cargos include: Corticorelin, Cosyntropin, Seractide, Sincalide, Protirelin, Sermorelin, Somatorelin, Tesamorelin, Secretin, Secretin (human), Secretin (porcine), Thymosin, Thyroid hormone, Thymalfasin, Thymopentin, Calcitonin, Salmon Calcitonin, Elcatonin, Human Calcitonin, Teriparatide, Atosiban, Carbetocin, Oxytocin, Buserelin, Ozempic, Gonadorelin, Goserelin, Histrelin, Leuprolide, Nafarelin, Triptorelin, Abarelix, Cetrorelix, Degarelix, Ganirelix, Depreotide, Edotreotide, Lanreotide, Octreotide, Pentetreotide, Somatostatin, Vapreotide, Argipressin, Desmopressin, Lypressin, Phenypressin, Terlipressin, Enfuvirtide, Ziconotide, Saralasin, Bivalirudin, Eptifibatide, Badp1, Carperitide, Nesiritide, aCT1, aCT11-I, aCT1-I, Angiotensin, ANP, BMP, Liraglutide, Ghrelin, ANP, Ac2-26, LL-37, FF/CAP18, VIP, PIPS, AcF, FeG, CNP, BNP, MOTS-c, Elampretide, SBS272, SBT-550, Icatibant, Exenatide, Liraglutide, Lixisenatide, Albiglutide, Dulaglutide, Semaglutide, Pramlintide, Linasclotide, Sinaspultide, Pasireotide, Teduglutide, Peginesatide, Pentagastrin, Cecropin, moricin, Insect defensin, drosomycin Diptericins, Metchnikowin, Ponericins, Jelleines, Apisimin, Pyrrhocoricin, Persulcatusin, Melittin, Apidaecin, drosocin, lebocin, attacin, gloverin. Gap19, Rotigaptide, Peptif, L2, JM, ACT017, CB1, AXT107 and other therapeutic or bioactive cargos in esterified and natural forms as described in WIPO Patent Application WO/2022/182782, which is incorporated herein by reference. Exemplary cargo nutrients could include, without limitation, minerals (e.g., potassium, sodium, chloride, magnesium, manganese, cobalt, molybdenum, calcium, copper, zinc, iodine, iron, chromium, fluoride, selenium, etc.), vitamins (vitamin A, E, D, C, K, etc.), creatine, ATP, ADP, AMP, Adenosine, sugars (e.g., glucose, fructose, mannose, galactose, lactose, etc.), hyaluronic acid, forms of Vitamin A (retinol and other forms), Vitamin C (L-Ascorbic Acid), Biotin, Niacin, Pantothenic acid, Omega 3 fatty acids, fats and fatty acids.

Exemplary hormones that could be used as cargo include, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), purines (e.g., ATP), pyrimidines (e.g., thymine), enzymes (e.g., creatine) and steroid hormones (e.g. estradiol, testosterone, tetrahydro testosterone, cortisol).

Exemplary immunomodulators that may be used as cargo include prednisone, azathioprine, 6-MP, cyclosporine, tacrolimus, methotrexate, interleukins (e.g., IL-2, IL-7, and IL-12), cytokines (e.g. interferons (e.g. IFN-a, IFN-b, IFN-e, IFN-K, IFN-co, and IFN-g), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Exemplary antipyretics include, but are not limited to, non-steroidal anti inflammatories (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Exemplary anxiolytics include, benzodiazepines (e.g., alprazolam, bromazepam, chlordiazepoxide, clonazepam, clorazepate, diazepam, flurazepam, lorazepam, oxazepam, temazepam, triazolam, and tofisopam), serotonergic antidepressants (e.g., selective serotonin reuptake inhibitors, tricyclic antidepressants, and monoamine oxidase inhibitors), temgicoluril, fabomotizole, selank, bromantane, emoxypine, azapirones, barbiturates, hydroxyzine, pregabalin, isovaleric acid, and beta blockers.

Exemplary antipsychotics that could be used as cargo include benperidol, bromperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, bifeprunox, bitopertin, brexpiprazole, cannabidiol, cariprazine, pimavanserin, pomaglumetad methionil, vabicaserin, xanomeline, and zicronapine.

Exemplary analgesics that may be used as cargo include paracetamol/acetaminophen, nonsteroidal anti-inflammatoires (e.g. ibuprofen, naproxen, ketoprofen, and nimesulide), COX-2 inhibitors (e.g., rofecoxib, celecoxib, and etoricoxib), opioids (e.g. morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Exemplary antispasmodics that may be used as cargo include mebeverine, papaverine, cyclobenzaprine, carisoprodol, orphenadrine, tizanidine, metaxalone, methocarbamol, chlorzoxazone, baclofen, dantrolene, baclofen, tizanidine, succinylcholine and dantrolene. Suitable anti-inflammatoires include, but are not limited to, prednisone, non-steroidal anti-inflammatoire.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives).

Exemplary anti-histamines that may be used as cargo include H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocetirizine, loratadine, meclizine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, ranitidine, and roxatidine), tritoqualine, catechin, cromoglicate, nedocromil, and p2-adrenergic agonists.

Exemplary anti-infectives that could be used as cargo include amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, albendazole, thiabendazole, oxamniquine), antifungals (e.g., azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafme, flucytosine, and polyenes (e.g., nystatin, and amphotericin b), antimalarial agents (e.g., pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g., aminosalicylates (e.g., amino salicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscamet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, abacavir, zidovudine, stavudine, emtricitabine, zalcitabine, telbivudine, simeprevir, boceprevir, telaprevir, lopinavir/ritonavir, fosamprenavir, darunavir, ritonavir, tipranavir, atazanavir, nelfmavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g., vancomycin, dalbavancin, oritavancin, and telavancin), glycylcy dines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, di cl oxacillin, and nafcillin), quinolones (e.g., lomefloxacin, norfloxacin, ofloxacin, moxifloxacin, ciprofloxacin, levofloxacin, Gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfasoxazole), tetracyclines (e.g., doxycycline, demeclocycline, minocycline, doxycycline/salicylic acid, doxy cy cline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

Exemplary chemotherapeutics that may be used as cargo include paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, bebnostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, dacarbazine, leuprobde, epirubicin, oxabplatin, asparaginase, estramustine, cetuximab, vismodegib, asparaginase Erwinia chrysanthemin, amifostine, etoposide, flutamide, toremifene, fulvestrant, letrozole, degarebx, pralatrexate, methotrexate, floxuridine, obinutuzumab, gemcitabine, afatinib, imatinib mesylate, carmustine, eribubn, trastuzumab, altretamine, topotecan, ponatinib, idarubicin, ifosfamide, ibrutinib, axitinib, interferon alfa-2a, gefitinib, romidepsin, ixabepilone, ruxobtinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, abtretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrebn, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorebn, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserebn, vorinostat, idelalisib, ceritinib, abiraterone, epothilone, tafluposide, azathioprine, doxifluridine, vindesine, and all-trans retinoic acid.

Suitable radiation sensitizers include, but are not limited to, 5-fluorouracil, platinum analogs (e.g., cisplatin, carboplatin, and oxaliplatin), gemcitabine, DNA topoisomerase I-targeting drugs (e.g., camptothecin derivatives (e.g., topotecan and irinotecan)), epidermal growth factor receptor blockade family agents (e.g., cetuximab, gefitinib), famesyltransferase inhibitors (e.g., L-778-123), COX-2 inhibitors (e.g., rofecoxib, celecoxib, and etoricoxib), bFGF and VEGF targeting agents (e.g., bevazucimab and thalidomide), NBTXR3, Nimoral, trans sodium crocetinate, NVX-108, and combinations thereof. See also e.g., Kvols, L. K., J Nucl Med 2005; 46:187S-190S.

Exemplary immunogens that may be used as carried as cargo or attached to the external surface of the isolated vesicles could include Keyhole Limpet Hemocyanin (KLH), Concholepas Concholepas Hemocyanin (CCH), (also Blue Carrier Immunogenic Protein), Bovine Serum Albumin (BSA), Ovalbumin (OVA), and antigens used to generate immune responses to pathogens causing disease including that causing diphtheria, tetanus, pertussis, measles, mumps, rubella, hepatitis A, hepatitis B, meningococcal disease (e.g., meningitis), human papillomavirus varicella, rabies, flu, rotoviral, HIV, malarial and coronaviral disease.

Exemplary polynucleotide modification systems that may be used as carried as cargo or attached to the external surface of the isolated vesicles could include without limitation, CRISPR-Cas systems, OMEGA systems, PRIME editing systems, base editors, meganucleases, zinc-finger nucleases, recombinases, TALE nucleases, CAST systems, Non-LTR retrotransposon systems, transposons, RNAi, antisense nucleic acids, etc. See e.g., Crooke S T, Liang X H, Baker B F, Crooke R M. Antisense technology: A review. J Biol Chem. 2021; 296:100416. doi:10.1016/j.jbc.2021.100416; Shmakov et al. (2015) “Discovery and Functional Characterization of Diverse Class 2 CRISPR-Cas Systems”, Molecular Cell, DOI: dx.doi.org/10.1016/j.molcel.2015.10.008; Peters et al., PNAS 114 (35) (2017); DOI: 10.1073/pnas.1709035114; Makarova et al. 2018. The CRISPR Journal, v. 1, n 5, FIG. 5; Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160; Moscou et al., Science 326:1501(2009 ); Boch et al., Science 326:1509-1512 (2009); and Zhang et al., Nature Biotechnology 29:149-153 (2011); Rees and Liu. 2018. Nat. Rev. Gent. 19(12): 770-788; Komor et al. 2016. Nature. 533:420-424; Nishida et al. 2016. Science. 353; Gaudeli et al. 2017. Nature. 551:464-471; Cox et al. 2017. Science 358:1019-1027; Levy et al. Nature Biomedical Engineering doi.org/10.1038/s41441-019-0505-5 (2019); Gorsuch et al. (2022). Targeting the hepatitis B cccdna with a sequence-specific arcus nuclease to eliminate hepatitis B virus in vivo. Molecular Therapy, 30(9), 2909-2922. doi.org/10.1016/j.ymthe.2022.05.013; Anzalone et al. 2019. Nature. 576:149-157, particularly at FIGS. 1b, 1c, related discussion, and Supplementary discussion; Anzalone AV, Gao XD, Podracky CJ, et al. Programmable deletion, replacement, integration and inversion of large DNA sequences with twin prime editing. Nat Biotechnol. 2022; 40(5): 731-740; Yarnall et al., Nat Biotechnol (2022). doi.org/10.1038/s41587-022-01527-4; Groth, A. C. and Calos, M. P. (2004) J. Mol. Biol. 335, 667-678; Lei, et al., FEBS Lett. 2018 Apr; 592(8): 1389-1399; Singh, et al., Attachment Site Selection and Identity in Bxb 1 Serine Integrase-Mediated Site-Specific Recombination, PLOS Genet. 2013 May;9(5):e1003490; and Gupta, et al., Nucleic Acids Res. 2007 May; 35(10): 3407-3419; Tou et al. bioRxiv 2022.01.07.475005, doi.org/10.1101/2022.01.07.475005; Klompe et al. Nature, doi: 10.1038/s 41586-019-1323; Strecker et al. Science. 10/1126/science. aax 9181 (2019); Christensen SM et al., RNA from the 5′ end of the R2 retrotransposon controls R2 protein binding to and cleavage of its DNA target site, Proc Natl Acad Sci U S A. 2006 November 21; 103(47): 17602-7; Eickbush TH et al, Integration, Regulation, and Long-Term Stability of R2 Retrotransposons, Microbiol Spectr. 2015 Apr;3(2): MDNA3-0011-2014. doi: 10.1128/microbiolspec. MDNA3-0011-2014; Han JS, Non-long terminal repeat (non-LTR) retrotransposons: mechanisms, recent developments, and unanswered questions, Mob DNA. 2010 May 12;1(1):15.doi:10.1186/1759-8753-1-15; Malik H S et al., The age and evolution of non-LTR retrotransposable elements, Mol Biol Evol. 1999 Jun; 16(6): 793-805; U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, 6,479,626, 8,163,514, 8,133,697, 8,021,867, 8,119,361, 8,119,381, 8,124,369, 8,129,134, 10,851,358; U.S. Patent Application Publication No. 2020/0239544, 2018/0346934; International Patent Application Publications WO 2018/213708, WO 2018/213726, WO 2014/093622, WO 2019/005884, WO 2019/005886, WO 2019/071048, WO 2016/106236, WO 2020/206231; WO 2021/138469, WO 2020/131862, WO 2021/257997, WO 2021/087394, WO 2022/147321, WO 2022/07682, WO 2022/150651, WO 2021/102042, WO 2022/173830.

In some embodiments, the exogenous cargo is present in the milk vesicles at any non-zero amount ranging from 0.1 to/or 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg per milligram of vesicle protein, or be any numerical value or subrange within any of these ranges.

In other embodiments therapeutic cargos, as listed in any embodiments herein, may be esterified to facilitate cargo loading, for example, by endogenous vesicle enzymes as described in WIPO Patent Application WO/2022/182782, which is incorporated herein by reference.

### Pharmaceutical, Therapeutic and Nutraceutical Preparations and Uses of Vesicles

Extracellular vesicles can be powerful tools for administering a therapeutic cargo to a subject due to the ability of vesicles to transport and protect biological signaling molecules to a subject together with unique ability of certain extracellular vesicle populations to cross tissue boundaries such as the cutaneous barrier, blood-brain-barrier. Extracellular vesicles also in certain situations can elude immune surveillance and are immunologically well-tolerated even when transferred autologously between individuals and species. Targeting extracellular vesicles to injured tissue using membrane cloaking and surface display. J Nanobiotechnology)-further heightening interest in their potential for translation to the clinic as a novel means for improving the safety of drug delivery. Accordingly, also described herein include pharmaceutical formulations that can contain an amount, effective amount, and/or least effective amount, and/or therapeutically effective amount of one or vesicles, such as cargo loaded milk vesicles, as described herein and a pharmaceutically acceptable carrier or excipient. Described in certain example embodiments herein are formulations that include extracellular vesicles, where the formulation is produced at least in part by any one of the methods of any one of the preceding paragraphs and/or described elsewhere herein, such as in the Working Examples below. Described in certain example embodiments herein are methods that include administering a formulation as described in any one of the previous paragraphs and/or elsewhere herein, such as the Working Examples below, to a subject. In some embodiments, the formulation administered to the subject includes milk vesicles, such as any of those described elsewhere herein and/or prepared by a method described elsewhere herein. In some embodiments, the milk vesicles are cargo loaded milk vesicles.

As used herein, the terms “treating”, and “treatment” can refer generally to obtaining a desired pharmacological and/or physiological effect. The effect can be, but does not necessarily have to be, prophylactic in terms of preventing or partially preventing a disease, symptom or condition thereof, such as cancer, inflammatory disease or disorder, and/or a mechanical or non-mechanical injury. The effect can be therapeutic in terms of a partial or complete cure of a disease, condition, symptom or adverse effect attributed to the disease, disorder, or condition. In some embodiments the effect can be the alleviation or intended prevention of undesirable symptoms. The term “treatment” as used herein covers any treatment of cancer, inflammatory disease or disorder, and/or a mechanical or non-mechanical injury, in a subject, particularly a human or non-human animal, such as a non-human mammal, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

In some embodiments the subject to which the vesicles or formulation thereof is administered has a disease or disorder. Exemplary diseases or disorders include, but are not limited to, any cancer, a viral infection, a bacterial infection, a parasite infection, a external and internal wounds and tissue injuries, cancer, ischemic and/or hypoxic injuries (e.g. myocardial infarction, ischemic wounds and/or stroke), multiple sclerosis, psoriasis, scleroderma, acne, eczema, or a disease of the skin and/or connective tissues, cardiac diseases or disorders, neurodegenerative diseases or disorders, neurological disorders, atherosclerosis, pathologies involving epithelial permeablization and/or neovascularization (e.g., angiogenesis or vasculogenesis), respiratory distress syndrome (RDS), reperfusion injuries, dermal vascular blemish or malformation, macular degeneration, neovascularization of choriocapillaries through Bruch's membrane, diabetic retinopathy, (inflammatory and inflammation-related diseases and disorders), radiation injury, acute radiation syndrome, radiation therapy side-effects, radiation dermatitis, as well as diseases resulting from high dose radiation including Acute Radiation Syndrome, Chronic Radiation Syndrome, Bile duct Cancer, Bone Cancer, Brain cancer, breast cancer, colon cancer, esophageal cancer, gall bladder cancer, liver cancer, lung cancer, pancreatic cancer, pharyngeal cancer, ovarian cancer, salivary gland cancer, intestinal cancer, stomach cancer, thyroid cancer, urinary tract cancer, leukemia, lyphomas, multiple myeloma or any other cancer, non-malignant thyroid nodular disease, parathyroid adenoma or posterior subcapsular cataracts.

Wounds can be chronic wounds or wounds that appear to not completely heal. Wounds that have not healed within three months, for example, are said to be chronic. Chronic wounds include, diabetic foot ulcers, ischemic, venous ulcers, venous leg ulcers, venous stasis, arterial, pressure, vasculitic, infectious, decubitis, trauma-induced, gangrenous and mixed ulcers. Chronic wounds include wounds that are characterized by and/or chronic inflammation, deficient and overprofuse granulation tissue differentiation and failure of re-epithelialization and wound closure and longer repair times. Chronic wounds can include ocular ulcers, including corneal ulcers. Use of the disclosed invention in wound healing and tissue regeneration can include in humans and agricultural, sports and pet animals.

Tissue injuries can result from, for example, a cut, scrape, compression wound, stretch injury, laceration wound, crush wound, bite wound, graze, bullet wound, explosion injury, body piercing, stab wound, surgical wound, surgical intervention, medical intervention, host rejection following cell, tissue or organ grafting, pharmaceutical effect, pharmaceutical side-effect, bed sore, radiation injury, radiation illness, cosmetic skin wound, internal organ injury, disease process (e.g., asthma, cancer), infection, infectious agent, developmental process, maturational process (e.g., acne), genetic abnormality, developmental abnormality, environmental toxin, allergen, scalp injury, facial injury, jaw injury, sex organ injury, joint injury, excretory organ injury, foot injury, finger injury, toe injury, bone injury, eye injury, corneal injury, muscle injury, adipose tissue injury, lung injury, airway injury, hernia, anus injury, piles, ear injury, skin injury, abdominal injury, retinal injury, eye injury, corneal injury, arm injury, leg injury, athletic injury, back injury, birth injury, premature birth injury, toxic bite, sting, injury to barrier function, injury to endothelial barrier function, injury to epithelial barrier function, tendon injury, ligament injury, heart injury, heart valve injury, vascular system injury, cartilage injury, lymphatic system injury, craniocerebral trauma, dislocation, esophageal perforation, fistula, nail injury, foreign body, fracture, frostbite, hand injury, heat stress disorder, laceration, neck injury, self-mutilation, shock, traumatic soft tissue injury, spinal cord injury, spinal injury, sprain, strain, tendon injury, ligament injury, cartilage injury, thoracic injury, tooth injury, trauma, nervous system injury, bum, bum wound, wind bum, sun bum, chemical bum, aging, aneurism, stroke, radiation injury, surgical radiation injury, digestive tract injury, infarct, or ischemic injury.

Cardiac diseases and disorders that may be treated by compositions and methods described herein can include, but are not limited to, myocardial infarction, cardio myopathies (e.g., hypertrophic cardiomyopathy), arrhythmias, congestive heart failure. The regenerative effects of the provided composition may result in beneficial changes in membrane excitability and ion transients of the heart. There are many different types of arrhythmias that can lead to abnormal function in the human heart. Arrhythmias include, but are not limited to bradycardias, tachycardias, altemans, automaticity defects, reentrant arrhythmias, fibrillation, AV nodal arrhythmias, atrial arrhythmias and triggered beats, Long QT syndrome, Short QT syndrome, Brugada syndrome, premature atrial Contractions, wandering Atrial pacemaker, Multifocal atrial tachycardia, Atrial flutter, Atrial fibrillation, Supraventricular tachycardia, AV nodal reentrant tachycardia is the most common cause of Paroxysmal Supraventricular Tachycardia, Junctional rhythm, Junctional tachycardia, Premature junctional complex, Wolff-Parkinson-White syndrome, Lown-Ganong-Levine syndrome, Premature Ventricular Contractions (PVC) sometimes called Ventricular Extra Beats, altemans and discordant altemans, Accelerated idioventricular rhythm, Monomorphic Ventricular tachycardia, Polymorphic ventricular tachycardia, Ventricular fibrillation, First degree heart block, which manifests as PR prolongation, Second degree heart block, Type 1 Second degree heart block, Type 2 Second degree heart block, Third degree heart block, and several accessory pathway disorders (e.g., Wolff-Parkinson- White syndrome (WPW)).

Neurodegenerative and neurological disorders include, but are not limited to dementia, Alzheimer's disease, Parkinson's disease and related PD-diseases, amyotrophic lateral sclerosis (ALS), motor neuron disease, schizophrenia, spinocerebellar ataxia, prion disease, Spinal muscular atrophy (SMA), multiple sclerosis, epilepsy and other seizure disorders, and Huntington's disease.

Inflammatory diseases and inflammatory-related diseases and disorders can be asthma, eczema, sinusitis, atherosclerosis, arthritis (including but not limited to rheumatoid arthritis), inflammatory bowel disease, cutaneous and systemic mastocytosis, psoriasis, and multiple sclerosis. As used herein, the term “inflammatory disorder” can include diseases or disorders which are caused, at least in part, or exacerbated, by inflammation, which is generally characterized by increased blood flow, edema, activation of immune cells (e.g., proliferation, cytokine production, or enhanced phagocytosis), heat, redness, swelling, pain and/or loss of function in the affected tissue or organ. The cause of inflammation can be due to physical damage, chemical substances, micro-organisms, tissue necrosis, cancer, or other agents or conditions.

Inflammatory disorders include acute inflammatory disorders, chronic inflammatory disorders, and recurrent inflammatory disorders. Acute inflammatory disorders are generally of relatively short duration, and last for from about a few minutes to about one to two days, although they can last several weeks. Characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or longer, and are associated histologically with the presence of lymphocytes and macrophages and with proliferation of blood vessels and connective tissue. Recurrent inflammatory disorders include disorders which recur after a period of time or which have periodic episodes. Some inflammatory disorders fall within one or more categories. Exemplary inflammatory disorders include but are not limited to atherosclerosis; arthritis; inflammation-promoted cancers; asthma; autoimmune uveitis; adoptive immune response; dermatitis; multiple sclerosis; diabetic complications; osteoporosis; Alzheimer's disease; cerebral malaria; hemorrhagic fever; autoimmune disorders; Crohn's disease, and inflammatory bowel disease. In some embodiments, the inflammatory disorder is an autoimmune disorder that, in some aspects, is selected from lupus, rheumatoid arthritis, and autoimmune encephalomyelitis.

In some embodiments, the inflammatory disorder is a brain-related inflammatory disorder. The term “brain-related inflammatory” disorder is used herein to refer to a subset of inflammatory disorders that are caused, at least in part, or originate or are exacerbated, by inflammation in the brain of a subject.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the cargo can optionally be present in the pharmaceutical formulation as a pharmaceutically acceptable salt. In some embodiments, the pharmaceutical formulation can include, such as an active ingredient, one or more milk vesicles, such as cargo loaded milk extracellular vesicles, described in greater detail elsewhere herein.

In some embodiments, the cargo is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavemous, intracavitary, intracerebral, intracistemal, intracorneal, intracoronal (dental), intracoronary, intracorporus cavemosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s) and/or cargos.

Where appropriate, one or more milk vesicles, such as cargo loaded milk vesicles, described in greater detail elsewhere herein can be provided to a subject in need thereof as an ingredient, such as an active ingredient or agent, in a pharmaceutical formulation. As such, also described are pharmaceutical formulations containing one or more milk vesicles, such as cargo loaded milk vesicles, described in greater detail elsewhere herein include a cargo that is in the form of a pharmaceutically acceptable salt. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed. In some embodiments the active agent is a milk vesicle, or a cargo loaded milk vesicle. In some embodiments, the active agent includes or is the cargo of a cargo loaded milk vesicle.

In certain embodiments, the milk vesicle is prepared by any method described elsewhere herein. In some embodiments, the cargo loaded milk vesicle is as described and/or prepared by a method as described elsewhere herein.

### Milk Vesicles

Mammalian milk is enriched in extracellular vesicles and has great promise as a source for large scale production extracellular vesicles for wide spread use as delivery vehicles. exemplary species used in milk production which can be used as a source of milk vesicles as provided herein include cows, buffalo, goats, sheep, camels, yaks, horses, reindeers, donkeys and humans. Bovine or cows milk is produced in large quantities by the dairy industry, widely consumed, and generally immunologically well-tolerated by humans. Moreover, milk vesicles have been reported to cross from the gut into the blood circulation and traffic to various organs, including brain, heart, gut and lungs (Wolf T, B. S. (2015). The intestinal transport of bovine milk vesicles is mediated by endocytosis in human colon carcinoma Caco-2 cells and rat small intestinal IEC-6 cells. J Nutr, 2201-2206), which are properties that could provide a basis for oral administration of cargo (including but not limited to therapeutics) loaded vesicles.

Milk used as a source for vesicles in the compositions and methods disclosed herein may be from any mammal. In some embodiments, the mammalian milk is bovine milk, ovine milk, porcine milk, camelid milk, equine milk, capra milk, human milk, and/or the like. In certain embodiments, the milk is unpasteurized. In some embodiments, the milk is pasteurized.

### Isolation and Purification of Extracellular Vesicles from Milk

Methods for large scale and cost-effective means for isolating extracellular vesicles from body fluids are of high interest for ultimate commercial scale use of vesicles as delivery vehicles. There are a number of methods for exosomal isolation from biological fluids, that are based on ultracentrifugation (UC). These methods typically involve differential centrifugation steps and/or density gradient UC-based separations. However, the ability to produce vesicles in large quantities is restricted by the requirement for multiple UC steps and the fact that UCs can only spin small volumes. It further remains that shearing forces imparted during repeated UC spins may have deleterious effects on vesicle structural integrity (Taylor DD, S. S. (2015). Methods of isolating extracellular vesicles impact down-stream analyses of their cargoes. Methods, 3-10). Other techniques that may exert less physical rigor during exosomal isolation include ultrafiltration, tangential flow filtration (TFF), size exclusion chromatography (SEC), and polyethylene glycol precipitation-based methods.

While milk is a promising source of commercial scale extracellular vesicles, milk has particular challenges in the isolation of the vesicles. Milk contains a diverse mixture of proteins, minerals, lipids, and other macromolecules and this complexity of the milk composition contributes to challenges to the purification of the extracellular vesicles. Casein proteins are a major constituent of milk, making up approximately 80% of all milk proteins. Caseins aggregate into large, colloidal complexes with calcium phosphate and other milk proteins to form what are referred to as casein micelles. These micelles are approximately 10 nm in diameter and can further coalesce into larger coagulated structures (Bhat, M. T. (2016). Casein Proteins: structural and functional aspects. Intech). Casein micelle aggregates are thought to bind to and ensnare vesicles via chemical interactions, impeding separation from contaminating milk proteins; observations that are confirmed by transmission electron microscopy (TEM) analysis of milk-derived exosomal preparations (Sedykh S. E., B. E. (2020). Milk Exosomes: Isolation, Biochemistry, Morphology, and Perspectives of Use. In C. J. De Bona A.G., Extracellular Vesicles and their importance in human health. Intech Open). As a consequence, present methods for isolation of high-purity extracellular vesicles from milk are limited by contaminating proteins, such as casein. A close association between extracellular vesicles and casein-rich strands of proteins isolated from milk has been noted (FIG. 1). These associations recall another association between extracellular vesicles and extracellular matrix that has been observed called extracellular matrix vesicles or matrix bound nanovesicles (MBNs). Without being bound by theory, it is contemplated that detaching or loosening vesicles by methods described herein, including via use of free tryptophan to destabilize chemical interaction between vesicles and matrices that can include extracellular, collagen, fibrin or casein matrices, can increase the yield of purified vesicles isolated from such sources. Further it is contemplated that in certain embodiments the addition of free tryptophan to vesicles isolated thus will aid with maintenance of structure and bioactivity of vesicles during procedures including lyophilization, sterilization and storage as described herein.

Methods, such as described in PCT WO2022182782, that include incorporating chelation of Ca2+and other divalent cations at specified temperatures result in high-yield separation of structurally and functionally intact vesicles from milk proteins. In some situations, casein micelle solubilization and vesicle separation steps can be included in a method with UC-based and/or TFF and SEC filtration steps for vesicle isolation thereby providing a basis for large-scale production of purified high quality vesicles from milk. Such methods involving chelating divalent cations with ethylenediaminetetraacetic acid (EDTA) and/or other chelator(s), including, but not limited to, 1,2-bis(o-aminophenoxy)ethane-N,N,N′,N′-tetraacetic acid (BAPTA), ethylene glycol-bis(o-aminoethyl ether)-N,N,N′,N′-tetraacetic acid (EGTA), sodium citrate, sodium tripolyphosphate, sodium hexametaphosphate, tetrasodium pyrophosphates, other phosphates and poly-phosphates and/or nitrophen are effective in methods for isolating extracellular vesicles, including without ultracentrifugation. EDTA chemically immobilized on insoluble substrates can be used to chelate divalent cations from solutions either in the presence of a semi-permeable membrane or not to prevent contamination of milk sample solution. However, in some situations, such as, for example uses of vesicles for nutraceutical uses, it may be desirable to avoid the use of chemicals such as EDTA that can be considered by some to be harsh.

Accordingly, it is one object of the present disclosure to develop methods similar to WO2022182782 that reduce, or avoid completely, the use of EDTA and/or similar chemicals that may be considered as harsh by using alternative agents instead. For example, in some embodiments of the present disclosure methods that are similar to those of PCT WO2022182782 are provided that utilize tryptophan and/or tryptophan analogues as alternatives to EDTA. Free tryptophan is naturally present in milk and colostrum at concentrations of up to 10 mg/ml and enhanced by dairy industry practices such as fermentation. Breast milk is the only source of tryptophan (TRP) in breast-fed infants (PMC5906556). Thus, rather being a contaminant, free tryptophan is thought an essential constituent of milk. Accordingly, provided herein include methods of isolating vesicles from a biological fluid such as milk that, for example, may include (a) centrifuging a biological fluid under conditions suitable to separate fats from one or more other components of the biological fluid; (b) removing the separated fats from the biological fluid; (c) after step (b) centrifuging the remaining biological fluid one or more times and skimming any noticeable separated fats after each centrifuging in step (c); (d) filtering the remaining biological fluid after step (c); (e) optionally performing one or more ultracentrifugation steps after (d); (f) contacting the resultant solution with tryptophan and/or or more tryptophan analogues at about 20-60 degrees Celsius for about 15-120 minutes. After step (f), tangential flow filtration to obtain a retenate can optionally be performed (step (g)). The retenate can then optionally be ultracentrifuged via one or more ultracentrifugation steps. The retenate can then optionally be fractionated via column separation. In some embodiments the method includes step (e) or step (g) but not both.

In some embodiments, step (f) is performed with tryptophan and/or one or more tryptophan analogues at a concentration between 10 μM and 1 mM. In some embodiments, step (f) is performed by contacting (e.g., incubating) the milk vesicles with tryptophan and/or or more tryptophan analogues at about 30-42 degrees Celsius for about 15-120 minutes.

In certain example embodiments, step (a) comprises centrifuging the biological fluid at about 2,500 relative centrifugal force (RCF). In certain example embodiment, (a) comprises centrifuging the biological fluid at about 2,000 rcf to about 3,000 rcf. In certain example embodiment, (a) comprises centrifuging the biological fluid at about 2000 rcf, 2010 rcf, 2020 rcf, 2030 rcf, 2040 rcf, 2050 rcf, 2060 rcf, 2070 rcf, 2080 rcf, 2090 rcf, 2100 rcf, 2110 rcf, 2120 rcf, 2130 rcf, 2140 rcf, 2150 rcf, 2160 rcf, 2170 rcf, 2180 rcf, 2190 rcf, 2200 rcf, 2210 rcf, 2220 rcf, 2230 rcf, 2240 rcf, 2250 rcf, 2260 rcf, 2270 rcf, 2280 rcf, 2290 rcf, 2300 rcf, 2310 rcf, 2320 rcf, 2330 rcf, 2340 rcf, 2350 rcf, 2360 rcf, 2370 rcf, 2380 rcf, 2390 rcf, 2400 rcf, 2410 rcf, 2420 rcf, 2430 rcf, 2440 rcf, 2450 rcf, 2460 rcf, 2470 rcf, 2480 rcf, 2490 rcf, 2500 rcf, 2510 rcf, 2520 rcf, 2530 rcf, 2540 rcf, 2550 rcf, 2560 rcf, 2570 rcf, 2580 rcf, 2590 rcf, 2600 rcf, 2610 rcf, 2620 rcf, 2630 rcf, 2640 rcf, 2650 rcf, 2660 rcf, 2670 rcf, 2680 rcf, 2690 rcf, 2700 rcf, 2710 rcf, 2720 rcf, 2730 rcf, 2740 rcf, 2750 rcf, 2760 rcf, 2770 rcf, 2780 rcf, 2790 rcf, 2800 rcf, 2810 rcf, 2820 rcf, 2830 rcf, 2840 rcf, 2850 rcf, 2860 rcf, 2870 rcf, 2880 rcf, 2890 rcf, 2900 rcf, 2910 rcf, 2920 rcf, 2930 rcf, 2940 rcf, 2950 rcf, 2960 rcf, 2970 rcf, 2980 rcf, 2990 rcf, to/or about 3000 ref. In certain embodiments, step (a) is repeated 1-3 times. In some embodiments, step (a) is repeated 1, 2, or 3 times.

In some embodiments, step (b) includes a first centrifugation followed by a second centrifugation. In certain example embodiments, the first centrifugation includes centrifuging the remaining biological fluid at about 14,500 rcf for about 60 minutes. In certain example embodiments, the first centrifugation includes centrifuging the remaining biological fluid at about 13,500 rcf to about 15,500 ref for about 45 to about 75 minutes. In some embodiments, the first centrifugation includes centrifuging the remaining biological fluid at about 13500 rcf, 13550 rcf, 13600 rcf, 13650 rcf, 13700 rcf, 13750 rcf, 13800 rcf, 13850 rcf, 13900 rcf, 13950 rcf, 14000 rcf, 14050 rcf, 14100 rcf, 14150 rcf, 14200 rcf, 14250 rcf, 14300 rcf, 14350 rcf, 14400 rcf, 14450 rcf, 14500 rcf, 14550 rcf, 14600 rcf, 14650 rcf, 14700 rcf, 14750 rcf, 14800 rcf, 14850 rcf, 14900 rcf, 14950 rcf, 15000 rcf, 15050 rcf, 15100 rcf, 15150 rcf, 15200 rcf, 15250 rcf, 15300 rcf, 15350 rcf, 15400 rcf, 15450 rcf, or at about 15500 ref for about 45 min, 46 min, 47 min, 48 min, 49 min, 50 min, 51 min, 52 min, 53 min, 54 min, 55 min, 56 min, 57 min, 58 min, 59 min, 60 min, 61 min, 62 min, 63 min, 64 min, 65 min, 66 min, 67 min, 68 min, 69 min, 70 min, 71 min, 72 min, 73 min, 74 min, or about 75 min. In some embodiments, the second centrifugation is performed on the biological fluid remaining after the first centrifugation and wherein the second centrifugation is performed at about 25,800 rcf for about 60 minutes. In certain example embodiments, the second centrifugation is performed on the biological fluid remaining after the first centrifugation and the second centrifugation is performed at about 24,800 to about 26,800 rcf for about 45 to about 75 minutes. In certain example embodiments, the second centrifugation is performed on the biological fluid remaining after the first centrifugation and the second centrifugation is performed at about 24800 rcf, 24850 rcf, 24900 rcf, 24950 rcf, 25000 rcf, 25050 rcf, 25100 rcf, 25150 rcf, 25200 rcf, 25250 rcf, 25300 rcf, 25350 rcf, 25400 rcf, 25450 rcf, 25500 rcf, 25550 rcf, 25600 rcf, 25650 rcf, 25700 rcf, 25750 rcf, 25800 rcf, 25850 rcf, 25900 rcf, 25950 rcf, 26000 rcf, 26050 rcf, 26100 rcf, 26150 rcf, 26200 rcf, 26250 rcf, 26300 rcf, 26350 rcf, 26400 rcf, 26450 rcf, 26500 rcf, 26550 rcf, 26600 rcf, 26650 rcf, 26700 rcf, 26750 rcf, 26800 ref for about 45 min, 46 min, 47 min, 48 min, 49 min, 50 min, 51 min, 52 min, 53 min, 54 min, 55 min, 56 min, 57 min, 58 min, 59 min, 60 min, 61 min, 62 min, 63 min, 64 min, 65 min, 66 min, 67 min, 68 min, 69 min, 70 min, 71 min, 72 min, 73 min, 74 min, or about 75 min. In certain example embodiments, the second centrifugation is repeated 1-3 times with each repetition being performed on the remaining biological fluid from the centrifugation immediately prior. In certain example embodiments, the second centrifugation is repeated 1, 2, or 3 times with each repetition being performed on the remaining biological fluid from the centrifugation immediately prior.

In certain example embodiments, step (d) includes filtering the remaining biological fluid through one or more filters in series ranging from about a 0.45 micron (μm) filter to about a 0.22 micron filter. In some embodiments, each filter in the series is independently selected from a 0.22 micron, 0.23 micron, 0.24 micron, 0.25 micron, 0.26 micron, 0.27 micron, 0.28 micron, 0.29 micron, 0.3 micron, 0.31 micron, 0.32 micron, 0.33 micron, 0.34 micron, 0.35 micron, 0.36 micron, 0.37 micron, 0.38 micron, 0.39 micron, 0.4 micron, 0.41 micron, 0.42 micron, 0.43 micron, 0.44 micron, or 0.45 micron filter. In some embodiments, the number of filters in series ranges 1-10, such as 1, 2, 3, 4, 5, 6, 7, 8, 9, or 10 filters in series. In some embodiments, all filters in the series are the same size cut off. In some embodiments, at least 2 filters in the series have the same size cut off. In some embodiments, at least 2 filters in the series have different size cut offs. In some embodiments, all the filters in the series have different size cut offs. In some embodiments, the size exclusion decreases from large to small along a series of filters. For example, in a series of 3 filters, the first filter can be a 0.45 micron filter, the second filter can be a 0.3 micron filter and the last filter can be a 0.22 filter. Other configurations of filters in series will be appreciated in view of the description herein. In some embodiments, the filters in series are all the same material. In some embodiments, the filters in series are all different materials. In some embodiments, at least 2 filters in the series are the same material. In some embodiments, at least 2 filters are made of different materials. Exemplary filters include, but are not limited to, membrane filters (e.g., polyethersulfone membrane filters, polyvinylidene fluoride membrane filters, cellulose membrane filters, mixed cellulose esters membrane filters, cellulose acetate membrane filters, cellulose nitrate membrane filters, polyamide membrane filters, polycarbonate membrane filters, polytetrafluoroethylene membrane filters, polypropylene membrane filters, nitrocellulose membrane filters, and/or the like), glass fiber or bead filters, and/or the like. In some embodiments, step (d) includes filtering the remaining biological fluid through an about 0.45 micron filter followed by filtering the remaining biological fluid through an about 0.22 micron filter.

In some embodiments of the aforementioned method, step (e) includes 2 or more serial ultracentrifugation steps, wherein each step is performed on the remaining biological fluid from the prior ultracentrifugation. In certain example embodiments, (e) includes 2-10 (e.g., 2, 3, 4, 5, 6, 7, 8, 9, or 10) serial ultracentrifugation steps, wherein each step is performed on the remaining biological fluid from the prior ultracentrifugation. In certain example embodiments, (e) includes an ultracentrifugation step performed at about 50,000 rcf, an ultracentrifugation step performed at about 70,000 rcf, an ultracentrifugation step performed at about 100,000 rcf, or any combination thereof. In certain example embodiments, (e) includes an ultracentrifugation step performed at about 45,000 to about 55,000 rcf, an ultracentrifugation step performed at about 65,000 to about 75,000 rcf, an ultracentrifugation step performed at about 90,000 to about 110,000 rcf, or any combination thereof. In certain example embodiments, (e) includes an ultracentrifugation step performed at about 45,000 to about 55,000 rcf (e.g., at about 45000 rcf, 45100 rcf, 45200 rcf, 45300 rcf, 45400 rcf, 45500 rcf, 45600 rcf, 45700 rcf, 45800 rcf, 45900 rcf, 46000 rcf, 46100 rcf, 46200 rcf, 46300 rcf, 46400 rcf, 46500 rcf, 46600 rcf, 46700 rcf, 46800 rcf, 46900 rcf, 47000 rcf, 47100 rcf, 47200 rcf, 47300 rcf, 47400 rcf, 47500 rcf, 47600 rcf, 47700 rcf, 47800 rcf, 47900 rcf, 48000 rcf, 48100 rcf, 48200 rcf, 48300 rcf, 48400 rcf, 48500 rcf, 48600 rcf, 48700 rcf, 48800 rcf, 48900 rcf, 49000 rcf, 49100 rcf, 49200 rcf, 49300 rcf, 49400 rcf, 49500 rcf, 49600 rcf, 49700 rcf, 49800 rcf, 49900 rcf, 50000 rcf, 50100 rcf, 50200 rcf, 50300 rcf, 50400 rcf, 50500 rcf, 50600 rcf, 50700 rcf, 50800 rcf, 50900 rcf, 51000 rcf, 51100 rcf, 51200 rcf, 51300 rcf, 51400 rcf, 51500 rcf, 51600 rcf, 51700 rcf, 51800 rcf, 51900 rcf, 52000 rcf, 52100 rcf, 52200 rcf, 52300 rcf, 52400 rcf, 52500 rcf, 52600 rcf, 52700 rcf, 52800 rcf, 52900 rcf, 53000 rcf, 53100 rcf, 53200 rcf, 53300 rcf, 53400 rcf, 53500 rcf, 53600 rcf, 53700 rcf, 53800 rcf, 53900 rcf, 54000 rcf, 54100 rcf, 54200 rcf, 54300 rcf, 54400 rcf, 54500 rcf, 54600 rcf, 54700 rcf, 54800 rcf, 54900 rcf, or at about 55000 ref), an ultracentrifugation step performed at about 65,000 to about 75,000 ref (e.g., at about 65000 rcf, 65100 rcf, 65200 rcf, 65300 rcf, 65400 rcf, 65500 rcf, 65600 rcf, 65700 rcf, 65800 rcf, 65900 rcf, 66000 rcf, 66100 rcf, 66200 rcf, 66300 rcf, 66400 rcf, 66500 rcf, 66600 rcf, 66700 rcf, 66800 rcf, 66900 rcf, 67000 rcf, 67100 rcf, 67200 rcf, 67300 rcf, 67400 rcf, 67500 rcf, 67600 rcf, 67700 rcf, 67800 rcf, 67900 rcf, 68000 rcf, 68100 rcf, 68200 rcf, 68300 rcf, 68400 rcf, 68500 rcf, 68600 rcf, 68700 rcf, 68800 rcf, 68900 rcf, 69000 rcf, 69100 rcf, 69200 rcf, 69300 rcf, 69400 rcf, 69500 rcf, 69600 rcf, 69700 rcf, 69800 rcf, 69900 rcf, 70000 rcf, 70100 rcf, 70200 rcf, 70300 rcf, 70400 rcf, 70500 rcf, 70600 rcf, 70700 rcf, 70800 rcf, 70900 rcf, 71000 rcf, 71100 rcf, 71200 rcf, 71300 rcf, 71400 rcf, 71500 rcf, 71600 rcf, 71700 rcf, 71800 rcf, 71900 rcf, 72000 rcf, 72100 rcf, 72200 rcf, 72300 rcf, 72400 rcf, 72500 rcf, 72600 rcf, 72700 rcf, 72800 rcf, 72900 rcf, 73000 rcf, 73100 rcf, 73200 rcf, 73300 rcf, 73400 rcf, 73500 rcf, 73600 rcf, 73700 rcf, 73800 rcf, 73900 rcf, 74000 rcf, 74100 rcf, 74200 rcf, 74300 rcf, 74400 rcf, 74500 rcf, 74600 rcf, 74700 rcf, 74800 rcf, 74900 rcf, or at about 75000 rcl), an ultracentrifugation step performed at about 90,000 to about 110,000 ref (e.g., 90000 rcf, 90100 rcf, 90200 rcf, 90300 rcf, 90400 rcf, 90500 rcf, 90600 rcf, 90700 rcf, 90800 rcf, 90900 rcf, 91000 rcf, 91100 rcf, 91200 rcf, 91300 rcf, 91400 rcf, 91500 rcf, 91600 rcf, 91700 rcf, 91800 rcf, 91900 rcf, 92000 rcf, 92100 rcf, 92200 rcf, 92300 rcf, 92400 rcf, 92500 rcf, 92600 rcf, 92700 rcf, 92800 rcf, 92900 rcf, 93000 rcf, 93100 rcf, 93200 rcf, 93300 rcf, 93400 rcf, 93500 rcf, 93600 rcf, 93700 rcf, 93800 rcf, 93900 rcf, 94000 rcf, 94100 rcf, 94200 rcf, 94300 rcf, 94400 rcf, 94500 rcf, 94600 rcf, 94700 rcf, 94800 rcf, 94900 rcf, 95000 rcf, 95100 rcf, 95200 rcf, 95300 rcf, 95400 rcf, 95500 rcf, 95600 rcf, 95700 rcf, 95800 rcf, 95900 rcf, 96000 rcf, 96100 rcf, 96200 rcf, 96300 rcf, 96400 rcf, 96500 rcf, 96600 rcf, 96700 rcf, 96800 rcf, 96900 rcf, 97000 rcf, 97100 rcf, 97200 rcf, 97300 rcf, 97400 rcf, 97500 rcf, 97600 rcf, 97700 rcf, 97800 rcf, 97900 rcf, 98000 rcf, 98100 rcf, 98200 rcf, 98300 rcf, 98400 rcf, 98500 rcf, 98600 rcf, 98700 rcf, 98800 rcf, 98900 rcf, 99000 rcf, 99100 rcf, 99200 rcf, 99300 rcf, 99400 rcf, 99500 rcf, 99600 rcf, 99700 rcf, 99800 rcf, 99900 rcf, 100000 rcf, 100100 rcf, 100200 rcf, 100300 rcf, 100400 rcf, 100500 rcf, 100600 rcf, 100700 rcf, 100800 rcf, 100900 rcf, 101000 rcf, 101100 rcf, 101200 rcf, 101300 rcf, 101400 rcf, 101500 rcf, 101600 rcf, 101700 rcf, 101800 rcf, 101900 rcf, 102000 rcf, 102100 rcf, 102200 rcf, 102300 rcf, 102400 rcf, 102500 rcf, 102600 rcf, 102700 rcf, 102800 rcf, 102900 rcf, 103000 rcf, 103100 rcf, 103200 rcf, 103300 rcf, 103400 rcf, 103500 rcf, 103600 rcf, 103700 rcf, 103800 rcf, 103900 rcf, 104000 rcf, 104100 rcf, 104200 rcf, 104300 rcf, 104400 rcf, 104500 rcf, 104600 rcf, 104700 rcf, 104800 rcf, 104900 rcf, 105000 rcf, 105100 rcf, 105200 rcf, 105300 rcf, 105400 rcf, 105500 rcf, 105600 rcf, 105700 rcf, 105800 rcf, 105900 rcf, 106000 rcf, 106100 rcf, 106200 rcf, 106300 rcf, 106400 rcf, 106500 rcf, 106600 rcf, 106700 rcf, 106800 rcf, 106900 rcf, 107000 rcf, 107100 rcf, 107200 rcf, 107300 rcf, 107400 rcf, 107500 rcf, 107600 rcf, 107700 rcf, 107800 rcf, 107900 rcf, 108000 rcf, 108100 rcf, 108200 rcf, 108300 rcf, 108400 rcf, 108500 rcf, 108600 rcf, 108700 rcf, 108800 rcf, 108900 rcf, 109000 rcf, 109100 rcf, 109200 rcf, 109300 rcf, 109400 rcf, 109500 rcf, 109600 rcf, 109700 rcf, 109800 rcf, 109900 rcf, or at about 110000 rcf), or any combination thereof. In certain example embodiments, the one or more of the one or more ultracentrifugation steps are each performed for about 60 minutes. In certain example embodiments, the one or more of the one or more ultracentrifugation steps are each performed for about 45-75 minutes. In certain example embodiments, the one or more of the one or more ultracentrifugation steps are each performed for about 45 min, 46 min, 47 min, 48 min, 49 min, 50 min, 51 min, 52 min, 53 min, 54 min, 55 min, 56 min, 57 min, 58 min, 59 min, 60 min, 61 min, 62 min, 63 min, 64 min, 65 min, 66 min, 67 min, 68 min, 69 min, 70 min, 71 min, 72 min, 73 min, 74 min, or about 75 min.

In some embodiments, step (e) comprises a final ultracentrifugation step performed at about 130,000 rcf for about 120 minutes, the resulting fluid is discarded, and the remaining pellet is resuspended in a suitable volume of a suitable solution prior to (1). In certain example embodiments, (e) comprises a final ultracentrifugation step performed at about 115,000 to about 145,000 rcf for about 90-150 minutes, the resulting fluid is discarded, and the remaining pellet is resuspended in a suitable volume of a suitable solution prior to (1). In certain example embodiments, (e) comprises a final ultracentrifugation step performed at about 115,000 to about 145,000 ref (e.g., 115000 rcf, 115100 rcf, 115200 rcf, 115300 rcf, 115400 rcf, 115500 rcf, 115600 rcf, 115700 rcf, 115800 rcf, 115900 rcf, 116000 rcf, 116100 rcf, 116200 rcf, 116300 rcf, 116400 rcf, 116500 rcf, 116600 rcf, 116700 rcf, 116800 rcf, 116900 rcf, 117000 rcf, 117100 rcf, 117200 rcf, 117300 rcf, 117400 rcf, 117500 rcf, 117600 rcf, 117700 rcf, 117800 rcf, 117900 rcf, 118000 rcf, 118100 rcf, 118200 rcf, 118300 rcf, 118400 rcf, 118500 rcf, 118600 rcf, 118700 rcf, 118800 rcf, 118900 rcf, 119000 rcf, 119100 rcf, 119200 rcf, 119300 rcf, 119400 rcf, 119500 rcf, 119600 rcf, 119700 rcf, 119800 rcf, 119900 rcf, 120000 rcf, 120100 rcf, 120200 rcf, 120300 rcf, 120400 rcf, 120500 rcf, 120600 rcf, 120700 rcf, 120800 rcf, 120900 rcf, 121000 rcf, 121100 rcf, 121200 rcf, 121300 rcf, 121400 rcf, 121500 rcf, 121600 rcf, 121700 rcf, 121800 rcf, 121900 rcf, 122000 rcf, 122100 rcf, 122200 rcf, 122300 rcf, 122400 rcf, 122500 rcf, 122600 rcf, 122700 rcf, 122800 rcf, 122900 rcf, 123000 rcf, 123100 rcf, 123200 rcf, 123300 rcf, 123400 rcf, 123500 rcf, 123600 rcf, 123700 rcf, 123800 rcf, 123900 rcf, 124000 rcf, 124100 rcf, 124200 rcf, 124300 rcf, 124400 rcf, 124500 rcf, 124600 rcf, 124700 rcf, 124800 rcf, 124900 rcf, 125000 rcf, 125100 rcf, 125200 rcf, 125300 rcf, 125400 rcf, 125500 rcf, 125600 rcf, 125700 rcf, 125800 rcf, 125900 rcf, 126000 rcf, 126100 rcf, 126200 rcf, 126300 rcf, 126400 rcf, 126500 rcf, 126600 rcf, 126700 rcf, 126800 rcf, 126900 rcf, 127000 rcf, 127100 rcf, 127200 rcf, 127300 rcf, 127400 rcf, 127500 rcf, 127600 rcf, 127700 rcf, 127800 rcf, 127900 rcf, 128000 rcf, 128100 rcf, 128200 rcf, 128300 rcf, 128400 rcf, 128500 rcf, 128600 rcf, 128700 rcf, 128800 rcf, 128900 rcf, 129000 rcf, 129100 rcf, 129200 rcf, 129300 rcf, 129400 rcf, 129500 rcf, 129600 rcf, 129700 rcf, 129800 rcf, 129900 rcf, 130000 rcf, 130100 rcf, 130200 rcf, 130300 rcf, 130400 rcf, 130500 rcf, 130600 rcf, 130700 rcf, 130800 rcf, 130900 rcf, 131000 rcf, 131100 rcf, 131200 rcf, 131300 rcf, 131400 rcf, 131500 rcf, 131600 rcf, 131700 rcf, 131800 rcf, 131900 rcf, 132000 rcf, 132100 rcf, 132200 rcf, 132300 rcf, 132400 rcf, 132500 rcf, 132600 rcf, 132700 rcf, 132800 rcf, 132900 rcf, 133000 rcf, 133100 rcf, 133200 rcf, 133300 rcf, 133400 rcf, 133500 rcf, 133600 rcf, 133700 rcf, 133800 rcf, 133900 rcf, 134000 rcf, 134100 rcf, 134200 rcf, 134300 rcf, 134400 rcf, 134500 rcf, 134600 rcf, 134700 rcf, 134800 rcf, 134900 rcf, 135000 rcf, 135100 rcf, 135200 rcf, 135300 rcf, 135400 rcf, 135500 rcf, 135600 rcf, 135700 rcf, 135800 rcf, 135900 rcf, 136000 rcf, 136100 rcf, 136200 rcf, 136300 rcf, 136400 rcf, 136500 rcf, 136600 rcf, 136700 rcf, 136800 rcf, 136900 rcf, 137000 rcf, 137100 rcf, 137200 rcf, 137300 rcf, 137400 rcf, 137500 rcf, 137600 rcf, 137700 rcf, 137800 rcf, 137900 rcf, 138000 rcf, 138100 rcf, 138200 rcf, 138300 rcf, 138400 rcf, 138500 rcf, 138600 rcf, 138700 rcf, 138800 rcf, 138900 rcf, 139000 rcf, 139100 rcf, 139200 rcf, 139300 rcf, 139400 rcf, 139500 rcf, 139600 rcf, 139700 rcf, 139800 rcf, 139900 rcf, 140000 rcf, 140100 rcf, 140200 rcf, 140300 rcf, 140400 rcf, 140500 rcf, 140600 rcf, 140700 rcf, 140800 rcf, 140900 rcf, 141000 rcf, 141100 rcf, 141200 rcf, 141300 rcf, 141400 rcf, 141500 rcf, 141600 rcf, 141700 rcf, 141800 rcf, 141900 rcf, 142000 rcf, 142100 rcf, 142200 rcf, 142300 rcf, 142400 rcf, 142500 rcf, 142600 rcf, 142700 rcf, 142800 rcf, 142900 rcf, 143000 rcf, 143100 rcf, 143200 rcf, 143300 rcf, 143400 rcf, 143500 rcf, 143600 rcf, 143700 rcf, 143800 rcf, 143900 rcf, 144000 rcf, 144100 rcf, 144200 rcf, 144300 rcf, 144400 rcf, 144500 rcf, 144600 rcf, 144700 rcf, 144800 rcf, 144900 rcf, or at about 145000 rcf) for about 90-150 minutes (e.g., 90 min, 91 min, 92 min, 93 min, 94 min, 95 min, 96 min, 97 min, 98 min, 99 min, 100 min, 101 min, 102 min, 103 min, 104 min, 105 min, 106 min, 107 min, 108 min, 109 min, 110 min, 111 min, 112 min, 113 min, 114 min, 115 min, 116 min, 117 min, 118 min, 119 min, 120 min, 121 min, 122 min, 123 min, 124 min, 125 min, 126 min, 127 min, 128 min, 129 min, 130 min, 131 min, 132 min, 133 min, 134 min, 135 min, 136 min, 137 min, 138 min, 139 min, 140 min, 141 min, 142 min, 143 min, 144 min, 145 min, 146 min, 147 min, 148 min, 149 min, or about 150 min), the resulting fluid is discarded, and the remaining pellet is resuspended in a suitable volume of a suitable solution prior to (1).

In certain example embodiments, the tangential flow filtration of (g) is performed using a 500 kDa ultrafiltration membrane. In certain example embodiments, the tangential flow filtration of (g) is performed using an ultrafiltration membrane with about a molecular weight cutoff ranging from about 250 kDa to about 750 kDa. In some embodiments the molecular weight cutoff of the ultrafiltration membrane with a molecular weight cutoff of about 250 kDa, 260 kDa, 270 kDa, 280 kDa, 290 kDa, 300 kDa, 310 kDa, 320 kDa, 330 kDa, 340 kDa, 350 kDa,460 kDa, 470 kDa, 480 kDa, 490 kDa, 500 kDa, 510 kDa, 520 kDa, 530 kDa, 540 kDa, 550 kDa,560 kDa, 570 kDa, 580 kDa, 590 kDa, 600 kDa, 610 kDa, 620 kDa, 630 kDa, 640 kDa, 650 kDa, 660 kDa, 670 kDa, 680 kDa, 690 kDa, 700 kDa, 710 kDa, 720 kDa, 730 kDa, 740 kDa, or about 750 k Da.

In certain example embodiments, the tangential flow filtration of step (g) is performed at a flow rate of about 10 mL per minute. In certain example embodiments, the tangential flow filtration of (g) is performed at a flow rate ranging from about 5 mL to about 15 mL per minute. In certain example embodiments, the tangential flow filtration of (g) is performed at a flow rate of about 5 mL/min, 5.5 mL/min, 6 mL/min, 6.5 mL/min, 7 mL/min, 7.5 mL/min, 8 mL/min, 8.5 mL/min, 9 mL/min, 9.5 mL/min, 10 mL/min, 10.5 mL/min, 11 mL/min, 11.5 mL/min, 12 mL/min, 12.5 mL/min, 13 mL/min, 13.5 mL/min, 14 mL/min, 14.5 mL/min, or about 15 mL/min. In certain example embodiments, in step (g), when the amount of remaining biological fluid reaches about ten percent of its starting volume before tangential flow filtration the retenate is diafiltered with a suitable buffer.

In certain example embodiments, the method further includes ultracentrifuging the retenate when the retenate reaches about 20 percent of the starting diafiltration amount. In certain example embodiments, the ultracentrifugation of the retenate is performed at about 130,000 rcf for about 120 at about 4 degrees Celsius. In certain example embodiments the ultracentrifugation of the retenate performed at about 115,000 to about 145,000 rcf (e.g., 115000 rcf, 115050 rcf, 115100 rcf, 115150 rcf, 115200 rcf, 115250 rcf, 115300 rcf, 115350 rcf, 115400 rcf, 115450 rcf, 115500 rcf, 115550 rcf, 115600 rcf, 115650 rcf, 115700 rcf, 115750 rcf, 115800 rcf, 115850 rcf, 115900 rcf, 115950 rcf, 116000 rcf, 116050 rcf, 116100 rcf, 116150 rcf, 116200 rcf, 116250 rcf, 116300 rcf, 116350 rcf, 116400 rcf, 116450 rcf, 116500 rcf, 116550 rcf, 116600 rcf, 116650 rcf, 116700 rcf, 116750 rcf, 116800 rcf, 116850 rcf, 116900 rcf, 116950 rcf, 117000 rcf, 117050 rcf, 117100 rcf, 117150 rcf, 117200 rcf, 117250 rcf, 117300 rcf, 117350 rcf, 117400 rcf, 117450 rcf, 117500 rcf, 117550 rcf, 117600 rcf, 117650 rcf, 117700 rcf, 117750 rcf, 117800 rcf, 117850 rcf, 117900 rcf, 117950 rcf, 118000 rcf, 118050 rcf, 118100 rcf, 118150 rcf, 118200 rcf, 118250 rcf, 118300 rcf, 118350 rcf, 118400 rcf, 118450 rcf, 118500 rcf, 118550 rcf, 118600 rcf, 118650 rcf, 118700 rcf, 118750 rcf, 118800 rcf, 118850 rcf, 118900 rcf, 118950 rcf, 119000 rcf, 119050 rcf, 119100 rcf, 119150 rcf, 119200 rcf, 119250 rcf, 119300 rcf, 119350 rcf, 119400 rcf, 119450 rcf, 119500 rcf, 119550 rcf, 119600 rcf, 119650 rcf, 119700 rcf, 119750 rcf, 119800 rcf, 119850 rcf, 119900 rcf, 119950 rcf, 120000 rcf, 120050 rcf, 120100 rcf, 120150 rcf, 120200 rcf, 120250 rcf, 120300 rcf, 120350 rcf, 120400 rcf, 120450 rcf, 120500 rcf, 120550 rcf, 120600 rcf, 120650 rcf, 120700 rcf, 120750 rcf, 120800 rcf, 120850 rcf, 120900 rcf, 120950 rcf, 121000 rcf, 121050 rcf, 121100 rcf, 121150 rcf, 121200 rcf, 121250 rcf, 121300 rcf, 121350 rcf, 121400 rcf, 121450 rcf, 121500 rcf, 121550 rcf, 121600 rcf, 121650 rcf, 121700 rcf, 121750 rcf, 121800 rcf, 121850 rcf, 121900 rcf, 121950 rcf, 122000 rcf, 122050 rcf, 122100 rcf, 122150 rcf, 122200 rcf, 122250 rcf, 122300 rcf, 122350 rcf, 122400 rcf, 122450 rcf, 122500 rcf, 122550 rcf, 122600 rcf, 122650 rcf, 122700 rcf, 122750 rcf, 122800 rcf, 122850 rcf, 122900 rcf, 122950 rcf, 123000 rcf, 123050 rcf, 123100 rcf, 123150 rcf, 123200 rcf, 123250 rcf, 123300 rcf, 123350 rcf, 123400 rcf, 123450 rcf, 123500 rcf, 123550 rcf, 123600 rcf, 123650 rcf, 123700 rcf, 123750 rcf, 123800 rcf, 123850 rcf, 123900 rcf, 123950 rcf, 124000 rcf, 124050 rcf, 124100 rcf, 124150 rcf, 124200 rcf, 124250 rcf, 124300 rcf, 124350 rcf, 124400 rcf, 124450 rcf, 124500 rcf, 124550 rcf, 124600 rcf, 124650 rcf, 124700 rcf, 124750 rcf, 124800 rcf, 124850 rcf, 124900 rcf, 124950 rcf, 125000 rcf, 125050 rcf, 125100 rcf, 125150 rcf, 125200 rcf, 125250 rcf, 125300 rcf, 125350 rcf, 125400 rcf, 125450 rcf, 125500 rcf, 125550 rcf, 125600 rcf, 125650 rcf, 125700 rcf, 125750 rcf, 125800 rcf, 125850 rcf, 125900 rcf, 125950 rcf, 126000 rcf, 126050 rcf, 126100 rcf, 126150 rcf, 126200 rcf, 126250 rcf, 126300 rcf, 126350 rcf, 126400 rcf, 126450 rcf, 126500 rcf, 126550 rcf, 126600 rcf, 126650 rcf, 126700 rcf, 126750 rcf, 126800 rcf, 126850 rcf, 126900 rcf, 126950 rcf, 127000 rcf, 127050 rcf, 127100 rcf, 127150 rcf, 127200 rcf, 127250 rcf, 127300 rcf, 127350 rcf, 127400 rcf, 127450 rcf, 127500 rcf, 127550 rcf, 127600 rcf, 127650 rcf, 127700 rcf, 127750 rcf, 127800 rcf, 127850 rcf, 127900 rcf, 127950 rcf, 128000 rcf, 128050 rcf, 128100 rcf, 128150 rcf, 128200 rcf, 128250 rcf, 128300 rcf, 128350 rcf, 128400 rcf, 128450 rcf, 128500 rcf, 128550 rcf, 128600 rcf, 128650 rcf, 128700 rcf, 128750 rcf, 128800 rcf, 128850 rcf, 128900 rcf, 128950 rcf, 129000 rcf, 129050 rcf, 129100 rcf, 129150 rcf, 129200 rcf, 129250 rcf, 129300 rcf, 129350 rcf, 129400 rcf, 129450 rcf, 129500 rcf, 129550 rcf, 129600 rcf, 129650 rcf, 129700 rcf, 129750 rcf, 129800 rcf, 129850 rcf, 129900 rcf, 129950 rcf, 130000 rcf, 130050 rcf, 130100 rcf, 130150 rcf, 130200 rcf, 130250 rcf, 130300 rcf, 130350 rcf, 130400 rcf, 130450 rcf, 130500 rcf, 130550 rcf, 130600 rcf, 130650 rcf, 130700 rcf, 130750 rcf, 130800 rcf, 130850 rcf, 130900 rcf, 130950 rcf, 131000 rcf, 131050 rcf, 131100 rcf, 131150 rcf, 131200 rcf, 131250 rcf, 131300 rcf, 131350 rcf, 131400 rcf, 131450 rcf, 131500 rcf, 131550 rcf, 131600 rcf, 131650 rcf, 131700 rcf, 131750 rcf, 131800 rcf, 131850 rcf, 131900 rcf, 131950 rcf, 132000 rcf, 132050 rcf, 132100 rcf, 132150 rcf, 132200 rcf, 132250 rcf, 132300 rcf, 132350 rcf, 132400 rcf, 132450 rcf, 132500 rcf, 132550 rcf, 132600 rcf, 132650 rcf, 132700 rcf, 132750 rcf, 132800 rcf, 132850 rcf, 132900 rcf, 132950 rcf, 133000 rcf, 133050 rcf, 133100 rcf, 133150 rcf, 133200 rcf, 133250 rcf, 133300 rcf, 133350 rcf, 133400 rcf, 133450 rcf, 133500 rcf, 133550 rcf, 133600 rcf, 133650 rcf, 133700 rcf, 133750 rcf, 133800 rcf, 133850 rcf, 133900 rcf, 133950 rcf, 134000 rcf, 134050 rcf, 134100 rcf, 134150 rcf, 134200 rcf, 134250 rcf, 134300 rcf, 134350 rcf, 134400 rcf, 134450 rcf, 134500 rcf, 134550 rcf, 134600 rcf, 134650 rcf, 134700 rcf, 134750 rcf, 134800 rcf, 134850 rcf, 134900 rcf, 134950 rcf, 135000 rcf, 135050 rcf, 135100 rcf, 135150 rcf, 135200 rcf, 135250 rcf, 135300 rcf, 135350 rcf, 135400 rcf, 135450 rcf, 135500 rcf, 135550 rcf, 135600 rcf, 135650 rcf, 135700 rcf, 135750 rcf, 135800 rcf, 135850 rcf, 135900 rcf, 135950 rcf, 136000 rcf, 136050 rcf, 136100 rcf, 136150 rcf, 136200 rcf, 136250 rcf, 136300 rcf, 136350 rcf, 136400 rcf, 136450 rcf, 136500 rcf, 136550 rcf, 136600 rcf, 136650 rcf, 136700 rcf, 136750 rcf, 136800 rcf, 136850 rcf, 136900 rcf, 136950 rcf, 137000 rcf, 137050 rcf, 137100 rcf, 137150 rcf, 137200 rcf, 137250 rcf, 137300 rcf, 137350 rcf, 137400 rcf, 137450 rcf, 137500 rcf, 137550 rcf, 137600 rcf, 137650 rcf, 137700 rcf, 137750 rcf, 137800 rcf, 137850 rcf, 137900 rcf, 137950 rcf, 138000 rcf, 138050 rcf, 138100 rcf, 138150 rcf, 138200 rcf, 138250 rcf, 138300 rcf, 138350 rcf, 138400 rcf, 138450 rcf, 138500 rcf, 138550 rcf, 138600 rcf, 138650 rcf, 138700 rcf, 138750 rcf, 138800 rcf, 138850 rcf, 138900 rcf, 138950 rcf, 139000 rcf, 139050 rcf, 139100 rcf, 139150 rcf, 139200 rcf, 139250 rcf, 139300 rcf, 139350 rcf, 139400 rcf, 139450 rcf, 139500 rcf, 139550 rcf, 139600 rcf, 139650 rcf, 139700 rcf, 139750 rcf, 139800 rcf, 139850 rcf, 139900 rcf, 139950 rcf, 140000 rcf, 140050 rcf, 140100 rcf, 140150 rcf, 140200 rcf, 140250 rcf, 140300 rcf, 140350 rcf, 140400 rcf, 140450 rcf, 140500 rcf, 140550 rcf, 140600 rcf, 140650 rcf, 140700 rcf, 140750 rcf, 140800 rcf, 140850 rcf, 140900 rcf, 140950 rcf, 141000 rcf, 141050 rcf, 141100 rcf, 141150 rcf, 141200 rcf, 141250 rcf, 141300 rcf, 141350 rcf, 141400 rcf, 141450 rcf, 141500 rcf, 141550 rcf, 141600 rcf, 141650 rcf, 141700 rcf, 141750 rcf, 141800 rcf, 141850 rcf, 141900 rcf, 141950 rcf, 142000 rcf, 142050 rcf, 142100 rcf, 142150 rcf, 142200 rcf, 142250 rcf, 142300 rcf, 142350 rcf, 142400 rcf, 142450 rcf, 142500 rcf, 142550 rcf, 142600 rcf, 142650 rcf, 142700 rcf, 142750 rcf, 142800 rcf, 142850 rcf, 142900 rcf, 142950 rcf, 143000 rcf, 143050 rcf, 143100 rcf, 143150 rcf, 143200 rcf, 143250 rcf, 143300 rcf, 143350 rcf, 143400 rcf, 143450 rcf, 143500 rcf, 143550 rcf, 143600 rcf, 143650 rcf, 143700 rcf, 143750 rcf, 143800 rcf, 143850 rcf, 143900 rcf, 143950 rcf, 144000 rcf, 144050 rcf, 144100 rcf, 144150 rcf, 144200 rcf, 144250 rcf, 144300 rcf, 144350 rcf, 144400 rcf, 144450 rcf, 144500 rcf, 144550 rcf, 144600 rcf, 144650 rcf, 144700 rcf, 144750 rcf, 144800 rcf, 144850 rcf, 144900 rcf, 144950 rcf, or about 145000 rcl) for about 90 minutes to about 150 minutes e.g., (about 90 min, 91 min, 92 min, 93 min, 94 min, 95 min, 96 min, 97 min, 98 min, 99 min, 100 min, 101 min, 102 min, 103 min, 104 min, 105 min, 106 min, 107 min, 108 min, 109 min, 110 min, 111 min, 112 min, 113 min, 114 min, 115 min, 116 min, 117 min, 118 min, 119 min, 120 min, 121 min, 122 min, 123 min, 124 min, 125 min, 126 min, 127 min, 128 min, 129 min, 130 min, 131 min, 132 min, 133 min, 134 min, 135 min, 136 min, 137 min, 138 min, 139 min, 140 min, 141 min, 142 min, 143 min, 144 min, 145 min, 146 min, 147 min, 148 min, 149 min, or about 150 min) at about 4 degrees Celsius.

In certain example embodiments, the retenate is not ultracentrifuged prior to optionally fractionating the retenate. In these embodiments, the retenate is stored at about -80 °C prior to fractionation that is optionally performed via column separation. Optional fractionating of the retenate can be performed via any suitable method, including but not limited to column separation (based on size, charge, affinity, avidity, or other method or separation strategy). Fractions containing the extracellular vesicles can be kept.

In certain example embodiments, the method yields a vesicle concentrate that is at least 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or at least 20 percent of the starting volume of biologic fluid, such as milk. In certain example embodiments, the method yields a vesicle concentrate that is about 7 percent, 7.5 percent, 8 percent, 8.5 percent, 9 percent, 9.5 percent, 10 percent, 10.5 percent, 11 percent, 11.5 percent, 12 percent, 12.5 percent, 13 percent, 13.5 percent, 14 percent, 14.5 percent, 15 percent, 15.5 percent, 16 percent, 16.5 percent, 17 percent, 17.5 percent, 18 percent, 18.5 percent, 19 percent, 19.5 percent, or about 20 percent, of the starting volume of biologic fluid, such as milk.

### Specific Contemplated Embodiments

In addition to the aspects and embodiments contemplated elsewhere herein, the following particular embodiments are specifically contemplated.

### ATP-Analogue-Extracellular Vesicle Embodiments

1. A composition comprising extracellular vesicles wherein said extracellular vesicles comprise between 0.01 and 1,000 μM ATP or an ATP analogue.
2. A composition comprising extracellular vesicles wherein said extracellular vesicles comprise between 0.1 and 100 μM ATP or an ATP analogue.
3. A composition comprising milk extracellular vesicles wherein said extracellular vesicles comprise between 0.01 and 1,000 μM ATP or an ATP analogue.
4. A composition comprising milk extracellular vesicles wherein said extracellular vesicles comprise between 0.1 and 100 μM ATP or an ATP analogue.
5. A composition comprising bovine milk extracellular vesicles wherein said extracellular vesicles comprise between 0.01 and 1,000 μM ATP or an ATP analogue.
6. A composition comprising bovine milk extracellular vesicles wherein said extracellular vesicles comprise between 0.1 and 100 μM ATP or an ATP analogue.
7. The composition of any of the preceding embodiments, wherein said extracellular vesicles comprise between 0.25 and 75 μM; 0.5 and 50 μM; or 0.75 and 25 μM; or 0.8 and 20 μM; or 0.85 and 15 μM; or 0.9 and 10 μM; or 0.5 and 5 μM; or 0.1 and 2 μM; or 0.5 and 1.5 MM; or 0.75 and 1.25 μM of ATP or an ATP analogue.
8. The composition of any of the preceding embodiments, wherein said extracellular vesicles comprise between 0.1 and 5 μM of ATP or an ATP analogue.
9. The composition of any of the preceding embodiments, wherein said extracellular vesicles comprise between 0.1 and 2 μM of ATP or an ATP analogue.
10. The composition of any of the preceding embodiments, wherein said extracellular vesicles comprise between 0.1 μM and 1 μM of ATP or an ATP analogue.
11. The composition of any of the preceding embodiments, wherein said extracellular vesicles comprise about 1 μM of ATP or an ATP analogue.
12. A composition comprising extracellular vesicles wherein said extracellular vesicles comprise between 1 ng and 1,000 μg ATP per mg extracellular vesicle protein.
13. A composition comprising extracellular vesicles wherein said extracellular vesicles comprise between 10 ng and 100 μg ATP per mg extracellular vesicle protein.
14. A composition comprising extracellular vesicles wherein said extracellular vesicles comprise between 100 ng and 10 μg ATP per mg extracellular vesicle protein.
15. A composition comprising extracellular vesicles wherein said extracellular vesicles comprise between 200 ng and 1 μg ATP per mg extracellular vesicle protein.
16. A composition comprising extracellular vesicles wherein said extracellular vesicles comprise between 300 ng and 750 ng ATP per mg extracellular vesicle protein.
17. A composition comprising extracellular vesicles wherein said extracellular vesicles comprise between 400 ng and 650 ng ATP per mg extracellular vesicle protein.
18. A composition comprising extracellular vesicles wherein said extracellular vesicles comprise between 450 ng and 600 ng ATP per mg extracellular vesicle protein.
19. A composition comprising extracellular vesicles wherein said extracellular vesicles comprise between 500 ng and 550 ng ATP per mg extracellular vesicle protein.

A composition comprising extracellular vesicles wherein said extracellular vesicles comprise about 1 ng per mg extracellular vesicle protein; 10 ng per mg extracellular vesicle protein; 100 ng per mg extracellular vesicle protein; 200 ng per mg extracellular vesicle protein; 300 ng per mg extracellular vesicle protein; 400 ng per mg extracellular vesicle protein; 450 ng per mg extracellular vesicle protein; 500 ng per mg extracellular vesicle protein; 550 ng per mg extracellular vesicle protein; 600 ng per mg extracellular vesicle protein; 650 ng per mg extracellular vesicle protein; 750 ng per mg extracellular vesicle protein; 1 μg per mg extracellular vesicle protein; 10 μg per mg extracellular vesicle protein; 100 μg per mg extracellular vesicle protein; or 1,000 μg per mg extracellular vesicle protein.
20. A composition comprising milk extracellular vesicles wherein said extracellular vesicles comprise between 1 ng and 1,000 μg ATP per mg extracellular vesicle protein.
21. A composition comprising milk extracellular vesicles wherein said extracellular vesicles comprise between 10 ng and 100 μg ATP per mg extracellular vesicle protein.
22 A composition comprising milk extracellular vesicles wherein said extracellular vesicles comprise between 100 ng and 10 μg ATP per mg extracellular vesicle protein.
23. A composition comprising milk extracellular vesicles wherein said extracellular vesicles comprise between 200 ng and 1 μg ATP per mg extracellular vesicle protein.
24. A composition comprising milk extracellular vesicles wherein said extracellular vesicles comprise between 300 ng and 750 ng ATP per mg extracellular vesicle protein.
25. A composition comprising milk extracellular vesicles wherein said extracellular vesicles comprise between 400 ng and 650 ng ATP per mg extracellular vesicle protein.
26. A composition comprising milk extracellular vesicles wherein said extracellular vesicles comprise between 450 ng and 600 ng ATP per mg extracellular vesicle protein.
27. A composition comprising milk extracellular vesicles wherein said extracellular vesicles comprise between 500 ng and 550 ng ATP per mg extracellular vesicle protein.
28. A composition comprising milk extracellular vesicles wherein said extracellular vesicles comprise about 1 ng per mg extracellular vesicle protein; 10 ng per mg extracellular vesicle protein; 100 ng per mg extracellular vesicle protein; 200 ng per mg extracellular vesicle protein; 300 ng per mg extracellular vesicle protein; 400 ng per mg extracellular vesicle protein; 450 ng per mg extracellular vesicle protein; 500 ng per mg extracellular vesicle protein; 550 ng per mg extracellular vesicle protein; 600 ng per mg extracellular vesicle protein; 650 ng per mg extracellular vesicle protein; 750 ng per mg extracellular vesicle protein; 1 μg per mg extracellular vesicle protein; 10 μg per mg extracellular vesicle protein; 100 μg per mg extracellular vesicle protein; or 1,000 μg per mg extracellular vesicle protein.
29. A composition comprising bovine milk extracellular vesicles wherein said extracellular vesicles comprise between 1 ng and 1,000 μg ATP per mg extracellular vesicle protein.
30. A composition comprising bovine milk extracellular vesicles wherein said extracellular vesicles comprise between 10 ng and 100 μg ATP per mg extracellular vesicle protein.
31 A composition comprising bovine milk extracellular vesicles wherein said extracellular vesicles comprise between 100 ng and 10 μg ATP per mg extracellular vesicle protein.
32. A composition comprising bovine milk extracellular vesicles wherein said extracellular vesicles comprise between 200 ng and 1 μg ATP per mg extracellular vesicle protein.
33. A composition comprising bovine milk extracellular vesicles wherein said extracellular vesicles comprise between 300 ng and 750 ng ATP per mg extracellular vesicle protein.
34. A composition comprising bovine milk extracellular vesicles wherein said extracellular vesicles comprise between 400 ng and 650 ng ATP per mg extracellular vesicle protein.
35 A composition comprising bovine milk extracellular vesicles wherein said extracellular vesicles comprise between 450 ng and 600 ng ATP per mg extracellular vesicle protein.
36 A composition comprising bovine milk extracellular vesicles wherein said extracellular vesicles comprise between 500 ng and 550 ng ATP per mg extracellular vesicle protein.
37. A composition comprising bovine milk extracellular vesicles wherein said extracellular vesicles comprise about 1 ng per mg extracellular vesicle protein; 10 ng per mg extracellular vesicle protein; 100 ng per mg extracellular vesicle protein; 200 ng per mg extracellular vesicle protein; 300 ng per mg extracellular vesicle protein; 400 ng per mg extracellular vesicle protein; 450 ng per mg extracellular vesicle protein; 500 ng per mg extracellular vesicle protein; 550 ng per mg extracellular vesicle protein; 600 ng per mg extracellular vesicle protein; 650 ng per mg extracellular vesicle protein; 750 ng per mg extracellular vesicle protein; 1 μg per mg extracellular vesicle protein; 10 μg per mg extracellular vesicle protein; 100 μg per mg extracellular vesicle protein; or 1,000 μg per mg extracellular vesicle protein.
38. The composition of any of the preceding embodiments, wherein said ATP or an ATP analogue comprises ATP.
39. The composition of any of the preceding embodiments, wherein said ATP or an ATP analogue comprises a non-hydrolyzable analogue of ATP.
40. The composition of any of the preceding embodiments, wherein said ATP or an ATP analogue comprises one or more selected from the group consisting of α,β-methylene-ATP (α,62 mATP); β,γmethylene-ATP (β, γmATP); 2-thio-ATP (2-SH-ATP); 2-methylthio-ATP (2-MeS-ATP); 2′,3′-O-2,4,6,-trinitrophenyl-ATP (TNP-ATP); 2′,3′-<9-(4-benzoyl)-ATP (BzATP); an N-alkyl-2 ATP; adenosine 5′-(β,γimido)triphosphate (AMP-PNP); ATP-MgCl2; 5-aminoimidazole-4-carboxamide ribonucleotide, disoproxil fumarate, ribavirin, azidothymidine, fludarabine, efavirenz and oxidized ATP (oATP), Adenosine, ADP, AMP, and other nucleotides (e.g. GTP, CTP, UTP, TTP).
41. The composition of any of the preceding embodiments, wherein said ATP or an ATP analogue comprises one or more selected from the group consisting Adenosine, ADP, and AMP.
42. The composition of any of the preceding embodiments, wherein said ATP or an ATP analogue consists essentially of one or more selected from the group consisting Adenosine, ADP, and AMP.
43.
44. The composition of any of the preceding embodiments, wherein the average particle size of the extracellular vesicles in said composition are between 10 and 1000 nm.
45. The composition of any of the preceding embodiments, wherein the average particle size of the extracellular vesicles in said composition is between 10 and 1000 nm, and wherein at least 75%, or 80%; or 90%, or 95%, or 98%, or 99% of the particles are between 10 and 1000 nm.
46. The composition of any of the preceding embodiments, wherein at least 75%, or 80%; or 90% or 95% of the particles are less than 600 nm.
47. The composition of any of the preceding embodiments, wherein at least 95% of the particles are less than 600 nm.
48. The composition of any of the preceding embodiments, wherein at least 99% of the particles are less than 600 nm.
49. The composition of any of the preceding embodiments, wherein at least 75%, or 80%; or 90%, or 95%, or 98%, or 99% of the particles are less than about 500 nm.
50. The composition of any of the preceding embodiments, wherein at least 95% of the particles are less than about 500 nm.
51. The composition of any of the preceding embodiments, wherein at least 99% of the particles are less than about 500 nm.
52. The composition of any of the preceding embodiments, wherein at least 75%, or 80%; or 90%, or 95%, or 98%, or 99% of the particles are less than about 250 nm.
53. The composition of any of the preceding embodiments, wherein at least 95% of the particles are less than about 250 nm.
54. The composition of any of the preceding embodiments, wherein at least 99% of the particles are less than about 250 nm.
55. The composition of any of the preceding embodiments, wherein at least 75%, or 80%; or 90%, or 95%, or 98%, or 99% of the particles are greater than 20 nm.
56. The composition of any of the preceding embodiments, wherein at least 95% of the particles are greater than 20 nm.
57. The composition of any of the preceding embodiments, wherein at least 99% of the particles are greater than 20 nm.
58. The composition of any of the preceding embodiments, wherein the average particle size of the extracellular vesicles in said composition is between 10 and 500 nm; or 10 and 300 nm; or 10 and 200 nm; or 10 and 185 nm, or 10 and 175 nm; or 10 and 170 nm; or 10 and 165 nm; or 10 and 160 nm; or 10 and 155 nm; or 10 and 150 nm; or 10 and 145 nm; or 10 and 140 nm; or 10 and 135 nm; or 10 and 130 nm; or 15 and 1,000 nm; or 15 and 500 nm; or 15 and 300 nm; or 15 and 200 nm; or 15 and 185 nm, or 15 and 175 nm; or 15 and 170 nm; or 15 and 165 nm; or 15 and 160 nm; or 15 and 155 nm; or 15 and 150 nm; or 15 and 145 nm; or 15 and 140 nm; or 15 and 135 nm; or 15 and 130 nm; or 20 and 1,000 nm; or 20 and 500 nm; or 20 and 300 nm; or 20 and 200 nm; or 20 and 185 nm, or 20 and 175 nm; or 20 and 170 nm; or 20 and 165 nm; or 20 and 160 nm; or 20 and 155 nm; or 20 and 150 nm; or 20 and 145 nm; or 20 and 140 nm; or 20 and 135 nm; or 20 and 130 nm; or 25 and 1,000 nm; or 25 and 500 nm; or 25 and 300 nm; or 25 and 200 nm; or 25 and 185 nm, or 25 and 175 nm; or 25 and 170 nm; or 25 and 165 nm; or 25 and 160 nm; or 25 and 155 nm; or 25 and 150 nm; or 25 and 145 nm; or 25 and 140 nm; or 25 and 135 nm; or 25 and 130 nm; or 30 and 1,000 nm; or 30 and 500 nm; or 30 and 300 nm; or 30 and 200 nm; or 30 and 185 nm, or 30 and 175 nm; or 30 and 170 nm; or 30 and 165 nm; or 30 and 160 nm; or 30 and 155 nm; or 30 and 150 nm; or 30 and 145 nm; or 30 and 140 nm; or 30 and 135 nm; or 30 and 130 nm; or 35 and 1,000 nm; or 35 and 500 nm; or 35 and 300 nm; or 35 and 200 nm; or 35 and 185 nm, or 35 and 175 nm; or 35 and 170 nm; or 35 and 165 nm; or 35 and 160 nm; or 35 and 155 nm; or 35 and 150 nm; or 35 and 145 nm; or 35 and 140 nm; or 35 and 135 nm; or 35 and 130 nm; or 40 and 1,000 nm; or 40 and 500 nm; or 40 and 300 nm; or 40 and 200 nm; or 40 and 185 nm, or 40 and 175 nm; or 40 and 170 nm; or 40 and 165 nm; or 40 and 160 nm; or 40 and 155 nm; or 40 and 150 nm; or 40 and 145 nm; or 40 and 140 nm; or 40 and 135 nm; or 40 and 130 nm; or 45 and 1,000 nm; or 45 and 500 nm; or 45 and 300 nm; or 45 and 200 nm; or 45 and 185 nm, or 45 and 175 nm; or 45 and 170 nm; or 45 and 165 nm; or 45 and 160 nm; or 45 and 155 nm; or 45 and 150 nm; or 45 and 145 nm; or 45 and 140 nm; or 45 and 135 nm; or 45 and 130 nm; or 50 and 1,000 nm; or 50 and 500 nm; or 50 and 300 nm; or 50 and 200 nm; or 50 and 185 nm, or 50 and 175 nm; or 50 and 170 nm; or 50 and 165 nm; or 50 and 160 nm; or 50 and 155 nm; or 50 and 150 nm; or 50 and 145 nm; or 50 and 140 nm; or 50 and 135 nm; or 50 and 130 nm; or 55 and 1,000 nm; or 55 and 500 nm; or 55 and 300 nm; or 55 and 200 nm; or 55 and 185 nm, or 55 and 175 nm; or 55 and 170 nm; or 55 and 165 nm; or 55 and 160 nm; or 55 and 155 nm; or 55 and 150 nm; or 55 and 145 nm; or 55 and 140 nm; or 55 and 135 nm; or 55 and 130 nm; or 60 and 1,000 nm; or 60 and 500 nm; or 60 and 300 nm; or 60 and 200 nm; or 60 and 185 nm, or 60 and 175 nm; or 60 and 170 nm; or 60 and 165 nm; or 60 and 160 nm; or 60 and 155 nm; or 60 and 150 nm; or 60 and 145 nm; or 60 and 140 nm; or 60 and 135 nm; or 60 and 130 nm; or 65 and 1,000 nm; or 65 and 500 nm; or 65 and 300 nm; or 65 and 200 nm; or 65 and 185 nm, or 65 and 175 nm; or 65 and 170 nm; or 65 and 165 nm; or 65 and 160 nm; or 65 and 155 nm; or 65 and 150 nm; or 65 and 145 nm; or 65 and 140 nm; or 65 and 135 nm; or 65 and 130 nm; or 70 and 1,000 nm; or 70 and 500 nm; or 70 and 300 nm; or 70 and 200 nm; or 70 and 185 nm, or 70 and 175 nm; or 70 and 170 nm; or 70 and 165 nm; or 70 and 160 nm; or 70 and 155 nm; or 70 and 150 nm; or 70 and 145 nm; or 70 and 140 nm; or 70 and 135 nm; or 70 and 130 nm; or 75 and 1,000 nm; or 75 and 500 nm; or 75 and 300 nm; or 75 and 200 nm; or 75 and 185 nm, or 75 and 175 nm; or 75 and 170 nm; or 75 and 165 nm; or 75 and 160 nm; or 75 and 155 nm; or 75 and 150 nm; or 75 and 145 nm; or 75 and 140 nm; or 75 and 135 nm; or 75 and 130 nm; or 90 and 1,000 nm; or 90 and 500 nm; or 90 and 300 nm; or 90 and 200 nm; or 90 and 185 nm, or 90 and 175 nm; or 90 and 170 nm; or 90 and 165 nm; or 90 and 160 nm; or 90 and 155 nm; or 90 and 150 nm; or 90 and 145 nm; or 90 and 140 nm; or 90 and 135 nm; or 90 and 130 nm; or 95 and 1,000 nm; or 95 and 500 nm; or 95 and 300 nm; or 95 and 200 nm; or 95 and 185 nm, or 95 and 175 nm; or 95 and 170 nm; or 95 and 165 nm; or 95 and 160 nm; or 95 and 155 nm; or 95 and 150 nm; or 95 and 145 nm; or 95 and 140 nm; or 95 and 135 nm; or 95 and 130 nm; or 100 and 1,000 nm; or 100 and 500 nm; or 100 and 300 nm; or 100 and 200 nm; or 100 and 185 nm, or 100 and 175 nm; or 100 and 170 nm; or 100 and 165 nm; or 100 and 150 nm; or 100 and 155 nm; or 100 and 150 nm; or 100 and 145 nm; or 100 and 100 nm; or 100 and 135 nm; or 100 and 130 nm; or 105 and 1,000 nm; or 105 and 500 nm; or 105 and 300 nm; or 105 and 200 nm; or 105 and 185 nm, or 105 and 175 nm; or 105 and 170 nm; or 105 and 165 nm; or 105 and 160 nm; or 105 and 155 nm; or 105 and 150 nm; or 105 and 145 nm; or 105 and 140 nm; or 105 and 135 nm; or 105 and 130 nm; or 110 and 1,000 nm; or 110 and 500 nm; or 110 and 300 nm; or 110 and 200 nm; or 110 and 185 nm, or 110 and 175 nm; or 110 and 170 nm; or 110 and 165 nm; or 110 and 150 nm; or 110 and 155 nm; or 110 and 150 nm; or 110 and 145 nm; or 110 and 100 nm; or 110 and 135 nm; or 110 and 130 nm; or 115 and 1,000 nm; or 115 and 500 nm; or 115 and 300 nm; or 115 and 200 nm; or 115 and 185 nm, or 115 and 175 nm; or 115 and 170 nm; or 115 and 165 nm; or 115 and 160 nm; or 115 and 155 nm; or 115 and 150 nm; or 115 and 145 nm; or 115 and 140 nm; or 115 and 135 nm; or 115 and 130 nm; or 120 and 1,000 nm; or 120 and 500 nm; or 120 and 300 nm; or 120 and 200 nm; or 120 and 185 nm, or 120 and 175 nm; or 120 and 170 nm; or 120 and 165 nm; or 120 and 150 nm; or 120 and 155 nm; or 120 and 150 nm; or 120 and 145 nm; or 120 and 100 nm; or 120 and 135 nm; or 120 and 130 nm.
59. The composition of any of the preceding embodiments, wherein the average particle size of the extracellular vesicles in said composition is between 50 and 500 nm.
60. The composition of any of the preceding embodiments, wherein the average particle size of the extracellular vesicles in said composition is between 100 and 200 nm.
61. The composition of any of the preceding embodiments, wherein the average particle size of the extracellular vesicles in said composition is between 100 and 200 nm, and wherein 95% of the extracellular vesicles have a particle size between 100 and 200 nm.
62. The composition of any of the preceding embodiments, wherein the average particle size of the extracellular vesicles in said composition is about 150 nm.
63. The composition of any of the preceding embodiments, wherein the average particle size of the extracellular vesicles in said composition is about 150 nm, and wherein 95% of the extracellular vesicles have a particle size between 100 and 200 nm.
64. The composition of any of the preceding embodiments wherein the extracellular vesicles are milk extracellular vesicles.
65 The composition of any of the preceding embodiments wherein the extracellular vesicles are bovine milk extracellular vesicles.
66. The composition of any of the preceding embodiments wherein the extracellular vesicles are bovine milk extracellular vesicles; and wherein the bovine milk extracellular vesicles comprise bovine IgG.
67. The composition of any of the preceding embodiments, wherein the extracellular vesicles are milk extracellular vesicles.
68. The composition of any one of the preceding embodiments, wherein the extracellular vesicles are milk extracellular vesicles, and wherein the extracellular vesicles are isolated from milk using a method of any of the “Isolation of Extracellular Vesicles From Milk” embodiments.
69. The composition of any of the preceding embodiments wherein the extracellular vesicles comprise one or more cargo molecules.
70. The composition of any of the preceding embodiments wherein the composition is a pharmaceutically acceptable composition.
71. A method of preparing an ATP-analogue-extracellular vesicle composition of any of the preceding embodiments, said method comprising contacting the extracellular vesicles with a solution comprising ATP and/or an ATP analogue to obtain a composition comprising ATP-analogue-extracellular vesicles.
72. A method of increasing uptake by cells, tissues and/or organs of a subject by administering to said subject extracellular vesicle composition of any of the preceding embodiments.
73. A method of treating a subject, comprising administering to said subject an extracellular vesicle composition of any of the preceding embodiments.
74. A method of treating a subject, comprising administering to said subject an extracellular vesicle composition of any of the preceding embodiments by oral, enteral, parenteral, intranasal, IV, topical or direct administration.
75. A method of increasing uptake of extracellular vesicles by dermal tissues of a subject, said method comprising: administering by topical application to skin an extracellular vesicle composition of any of the preceding embodiments in said subject.
76. A method of providing benefit in a healthy subject by administering extracellular vesicles loaded with one or more nutraceutical or cosmetical cargos (e.g. peptides, curcumin, flavonoids, amino acids, lipids, fatty acids, calcium, vitamins, ATP, creatine and so on) of a composition of any of the preceding embodiments to said subject.
77. A method of enhancing cargo loading of extracellular vesicles with an external cargo molecule including a peptide, small drug molecule, or other cargo compound.

### Isolation of Extracellular Vesicles From Milk Embodiments

1. A method of isolating extracellular vesicles from a milk sample, wherein the method involves contacting (or incubating) the milk with tryptophan and/or one or more tryptophan analogues and subsequently separating the milk extracellular vesicles from one or more milk components (such as for example casein-containing aggregates).
2. A method of isolating extracellular vesicles from a milk sample, wherein the method comprises contacting (incubating) the milk extracellular vesicles with tryptophan and/or or more tryptophan analogues at a concentration between 10 μM and 1 mM.
3. A method of isolating extracellular vesicles from a milk sample, wherein the method comprises contacting (incubating) the milk extracellular vesicles with tryptophan and/or or more tryptophan analogues at a temperature between 10 and 50 degrees Celsius for at least 10 minutes.
4. A method of isolating extracellular vesicles from a milk sample, wherein the method comprises contacting (incubating) the milk extracellular vesicles with tryptophan and/or more tryptophan analogues at a concentration between 10 μM and 1 mM at a temperature between 10 and 50 degrees Celsius for at least 10 minutes.
5. A method of isolating extracellular vesicles from a milk sample, wherein the method comprises contacting (incubating) the milk extracellular vesicles with tryptophan and/or more tryptophan analogues at a concentration between 10 M and 1 mM at a temperature between 10 and 50 degrees Celsius for between 10 and 60 minutes.
6. A method of isolating extracellular vesicles from a milk sample, wherein the method comprises contacting (incubating) the milk extracellular vesicles with tryptophan and/or more tryptophan analogues and/or Trehalose and/or Trehalose analogues at a concentration between 1 mM and 100 mM at a concentration between 10 μM and 1 mM at a temperature between 10 and 50 degrees Celsius for at least 10 minutes.
7. A method of isolating extracellular vesicles from a milk sample, wherein the method comprises contacting (incubating) the milk extracellular vesicles with tryptophan and/or more tryptophan analogues at a concentration between 10 μM and 1 mM and/or Trehalose and/or Trehalose analogues at a concentration between 1 mM and 100 mM at a temperature between 10 and 50 degrees Celsius for between 10 and 60 minutes.
8. A method of isolating extracellular vesicles from a milk sample, wherein the method comprises contacting (incubating) the milk extracellular vesicles with tryptophan and/or more tryptophan analogues at a concentration between 10 μM and 1 mM in conjunction with acid treatment to a pH of 4.6 at a temperature between 10 and 50 degrees Celsius for between 10 and 60 minutes.
9. A method of isolating extracellular vesicles from a milk sample, wherein the method comprises contacting (incubating) the milk extracellular vesicles with tryptophan and/or more tryptophan analogues at a concentration between 10 μM and 1 mM in conjunction with acid treatment to a pH between 2.0 and 6.0 at a temperature between 10 and 50 degrees Celsius for between 10 and 60 minutes.
10. A method of isolating extracellular vesicles from a milk sample, wherein the method comprises contacting (incubating) the milk extracellular vesicles with tryptophan and/or more tryptophan analogues at a concentration between 10 μM and 1 mM and/or Trehalose and/or Trehalose analogues at a concentration between 1 mM and 100 mM in conjunction with acid treatment to a pH of 4.6 at a temperature between 10 and 50 degrees Celsius for between 10 and 60 minutes.
11. A method of isolating extracellular vesicles from a milk sample, wherein the method comprises contacting (incubating) the milk extracellular vesicles with tryptophan and/or more tryptophan analogues at a concentration between 10 M and 1 mM and/or Trehalose and/or Trehalose analogues at a concentration between 1 mM and 100 mM in conjunction with acid treatment to a pH between 2.0 and 6.0 at a temperature between 10 and 50 degrees Celsius for between 10 and 60 minutes.
12. A method of isolating extracellular vesicles from a milk sample, wherein the method comprises contacting (incubating) the milk extracellular vesicles with tryptophan and/or more tryptophan analogues at a concentration between 10 μM and 1 mM in conjunction with Chymosin treatment at a temperature between 10 and 50 degrees Celsius for between 10 and 60 minutes
13. A method of isolating extracellular vesicles from a milk sample, wherein the method
comprises contacting (incubating) the milk extracellular vesicles with tryptophan and/or or more tryptophan analogues at a temperature between 10 and 80 degrees Celsius for at least 10 seconds.
14. A method of isolating extracellular vesicles from a milk sample, wherein the method
comprises contacting (incubating) the milk extracellular vesicles with tryptophan and/or or more tryptophan analogues at a concentration between 10 μM and 1 mM at a temperature between 10 and 80 degrees Celsius for at least 10 seconds.
15. A method of isolating extracellular vesicles from a milk sample, wherein the method comprises contacting (incubating) the milk extracellular vesicles with tryptophan and/or or more tryptophan analogues at a concentration between 10 μM and 1 mM at a temperature between 10 and 80 degrees Celsius for between 10 seconds and 60 minutes.
16. A method of isolating extracellular vesicles from a milk sample, wherein the method
comprises contacting (incubating) the milk extracellular vesicles with tryptophan and/or or more tryptophan analogues at a concentration between 10 μM and 1 mM and/or Trehalose and/or Trehalose analogues at a concentration between 1 mM and 100 mM at a temperature between 10 and 80 degrees Celsius for at least 10 seconds.
17. A method of isolating extracellular vesicles from a milk sample, wherein the method comprises contacting (incubating) the milk extracellular vesicles with tryptophan and/or or more tryptophan analogues at a concentration between 10 μM and 1 mM and/or Trehalose and/or Trehalose analogues at a concentration between 1 mM and 100 mM at a temperature between 10 and 80 degrees Celsius for between 10 seconds and 60 minutes.
18. A method of isolating extracellular vesicles from a milk sample, wherein the method comprises contacting (incubating) the milk extracellular vesicles with tryptophan and/or more tryptophan analogues at a concentration between 10 μM and 1 mM in conjunction with acid treatment to a pH of 4.6 at a temperature between 10 and 80 degrees Celsius for between 10 seconds and 60 minutes.
19. A method of isolating extracellular vesicles from a milk sample, wherein the method comprises contacting (incubating) the milk extracellular vesicles with tryptophan and/or more tryptophan analogues at a concentration between 10 μM and 1 mM in conjunction with acid treatment to a pH between 2.0 and 6.0 at a temperature between 10 and 80 degrees Celsius for between 10 seconds and 60 minutes.
20. A method of isolating extracellular vesicles from a milk sample, wherein the method comprises contacting (incubating) the milk extracellular vesicles with tryptophan and/or more tryptophan analogues at a concentration between 10 μM and 1 mM and/or Trehalose and/or Trehalose analogues at a concentration between 1 mM and 100 mM in conjunction with acid treatment to a pH of 4.6 at a temperature between 10 and 80 degrees Celsius for between 10 seconds and 60 minutes.
21. A method of isolating extracellular vesicles from a milk sample, wherein the method comprises contacting (incubating) the milk extracellular vesicles with tryptophan and/or more tryptophan analogues at a concentration between 10 μM and 1 mM and/or Trehalose and/or Trehalose analogues at a concentration between 1 mM and 100 mM in conjunction with acid treatment to a pH between 2.0 and 6.0 at a temperature between 10 and 80 degrees Celsius for between 10 seconds and 60 minutes.
22. A method of isolating extracellular vesicles from a milk sample, wherein the method comprises contacting (incubating) the milk extracellular vesicles with tryptophan and/or more tryptophan analogues at a concentration between 10 μM and 1 mM in conjunction with Chymosin treatment at a temperature between 10 and 80 degrees Celsius for between 10 seconds and 60 minutes.
23. A method of isolating extracellular vesicles from a milk sample, wherein the method comprises contacting (incubating) the milk extracellular vesicles with tryptophan and/or more tryptophan analogues and/or Trehalose and/or Trehalose analogues at a temperature between 10 and 60 degrees Celsius for at least 10 minutes.
24. A method of isolating extracellular vesicles from a milk sample, wherein the method
comprises contacting (incubating) the milk extracellular vesicles with tryptophan and/or or more tryptophan analogues at a concentration between 10 μM and 1 mM at a temperature between 10 and 60 degrees Celsius for at least 10 minutes.
25. A method of isolating extracellular vesicles from a milk sample, wherein the method comprises contacting (incubating) the milk extracellular vesicles with tryptophan and/or or more tryptophan analogues at a concentration between 10 AM and 1 mM at a temperature between 10 and 60 degrees Celsius for between 10 and 60 minutes.
26. A method of isolating extracellular vesicles from a milk sample, wherein the method
comprises contacting (incubating) the milk extracellular vesicles with tryptophan and/or or more tryptophan analogues at a concentration between 10 μM and 1 mM and/or Trehalose and/or Trehalose analogues at a concentration between 1 mM and 100 mM at a temperature between 10 and 60 degrees Celsius for at least 10 minutes.
27. A method of isolating extracellular vesicles from a milk sample, wherein the method comprises contacting (incubating) the milk extracellular vesicles with tryptophan and/or or more tryptophan analogues at a concentration between 10 μM and 1 mM and/or Trehalose and/or Trehalose analogues at a concentration between 1 mM and 100 mM at a temperature between 10 and 60 degrees Celsius for between 10 and 60 minutes.
28. A method of isolating extracellular vesicles from a milk sample, wherein the method comprises contacting (incubating) the milk extracellular vesicles with tryptophan and/or more tryptophan analogues at a concentration between 10 μM and 1 mM in conjunction with acid treatment to a pH of 4.6 at a temperature between 10 and 60 degrees Celsius for between 10 and 60 minutes.
29. A method of isolating extracellular vesicles from a milk sample, wherein the method comprises contacting (incubating) the milk extracellular vesicles with tryptophan and/or more tryptophan analogues at a concentration between 10 μM and 1 mM in conjunction with acid treatment to a pH between 2.0 and 6.0 at a temperature between 10 and 60 degrees Celsius for between 10 and 60 minutes.
30. A method of isolating extracellular vesicles from a milk sample, wherein the method comprises contacting (incubating) the milk extracellular vesicles with tryptophan and/or more tryptophan analogues at a concentration between 10 μM and 1 mM and/or Trehalose and/or Trehalose analogues at a concentration between 1 mM and 100 mM in conjunction with acid treatment to a pH of 4.6 at a temperature between 10 and 60 degrees Celsius for between 10 and 60 minutes.
31. A method of isolating extracellular vesicles from a milk sample, wherein the method comprises contacting (incubating) the milk extracellular vesicles with tryptophan and/or more tryptophan analogues at a concentration between 10 μM and 1 mM and/or Trehalose and/or Trehalose analogues at a concentration between 1 mM and 100 mM in conjunction with acid treatment to a pH between 2.0 and 6.0 at a temperature between 10 and 60 degrees Celsius for between 10 and 60 minutes.
32. A method of isolating extracellular vesicles from a milk sample, wherein the method comprises contacting (incubating) the milk extracellular vesicles with tryptophan and/or more tryptophan analogues at a concentration between 10 μM and 1 mM in conjunction with Chymosin treatment at a temperature between 10 and 60 degrees Celsius for between 10 and 60 minutes.
33. The method of any of the preceding embodiments, wherein the average particle size of the extracellular vesicles in said composition are between 10 and 1000 nm.
34. The method of any of the preceding embodiments, wherein the average particle size of the extracellular vesicles in said composition is between 10 and 1000 nm, and wherein at least 75%, or 80%; or 90%, or 95%, or 98%, or 99% of the particles are between 10 and 1000 nm.
35. The method of any of the preceding embodiments, wherein at least 75%, or 80%; or 90% or 95% of the particles are less than 600 nm.
36. The method of any of the preceding embodiments, wherein at least 95% of the particles are less than 600 nm.
37. The method of any of the preceding embodiments, wherein at least 99% of the particles are less than 600 nm.
38. The method of any of the preceding embodiments, wherein at least 75%, or 80%; or 90%, or 95%, or 98%, or 99% of the particles are less than about 500 nm.
39. The method of any of the preceding embodiments, wherein at least 95% of the particles are less than about 500 nm.
40. The method of any of the preceding embodiments, wherein at least 99% of the particles are less than about 500 nm.
41. The method of any of the preceding embodiments, wherein at least 75%, or 80%; or 90%, or 95%, or 98%, or 99% of the particles are less than about 250 nm.
42. The method of any of the preceding embodiments, wherein at least 95% of the particles are less than about 250 nm.
43. The method of any of the preceding embodiments, wherein at least 99% of the particles are less than about 250 nm.
44. The method of any of the preceding embodiments, wherein at least 75%, or 80%; or 90%, or 95%, or 98%, or 99% of the particles are greater than 20 nm.
45. The method of any of the preceding embodiments, wherein at least 95% of the particles are greater than 20 nm.
46. The method of any of the preceding embodiments, wherein at least 99% of the particles are greater than 20 nm.
47. The method of any of the preceding embodiments, wherein the average particle size of the extracellular vesicles in said composition is between 10 and 500 nm; or 10 and 300 nm; or 10 and 200 nm; or 10 and 185 nm, or 10 and 175 nm; or 10 and 170 nm; or 10 and 165 nm; or 10 and 160 nm; or 10 and 155 nm; or 10 and 150 nm; or 10 and 145 nm; or 10 and 140 nm; or 10 and 135 nm; or 10 and 130 nm; or 15 and 1,000 nm; or 15 and 500 nm; or 15 and 300 nm; or 15 and 200 nm; or 15 and 185 nm, or 15 and 175 nm; or 15 and 170 nm; or 15 and 165 nm; or 15 and 160 nm; or 15 and 155 nm; or 15 and 150 nm; or 15 and 145 nm; or 15 and 140 nm; or 15 and 135 nm; or 15 and 130 nm; or 20 and 1,000 nm; or 20 and 500 nm; or 20 and 300 nm; or 20 and 200 nm; or 20 and 185 nm, or 20 and 175 nm; or 20 and 170 nm; or 20 and 165 nm; or 20 and 160 nm; or 20 and 155 nm; or 20 and 150 nm; or 20 and 145 nm; or 20 and 140 nm; or 20 and 135 nm; or 20 and 130 nm; or 25 and 1,000 nm; or 25 and 500 nm; or 25 and 300 nm; or 25 and 200 nm; or 25 and 185 nm, or 25 and 175 nm; or 25 and 170 nm; or 25 and 165 nm; or 25 and 160 nm; or 25 and 155 nm; or 25 and 150 nm; or 25 and 145 nm; or 25 and 140 nm; or 25 and 135 nm; or 25 and 130 nm; or 30 and 1,000 nm; or 30 and 500 nm; or 30 and 300 nm; or 30 and 200 nm; or 30 and 185 nm, or 30 and 175 nm; or 30 and 170 nm; or 30 and 165 nm; or 30 and 160 nm; or 30 and 155 nm; or 30 and 150 nm; or 30 and 145 nm; or 30 and 140 nm; or 30 and 135 nm; or 30 and 130 nm; or 35 and 1,000 nm; or 35 and 500 nm; or 35 and 300 nm; or 35 and 200 nm; or 35 and 185 nm, or 35 and 175 nm; or 35 and 170 nm; or 35 and 165 nm; or 35 and 160 nm; or 35 and 155 nm; or 35 and 150 nm; or 35 and 145 nm; or 35 and 140 nm; or 35 and 135 nm; or 35 and 130 nm; or 40 and 1,000 nm; or 40 and 500 nm; or 40 and 300 nm; or 40 and 200 nm; or 40 and 185 nm, or 40 and 175 nm; or 40 and 170 nm; or 40 and 165 nm; or 40 and 160 nm; or 40 and 155 nm; or 40 and 150 nm; or 40 and 145 nm; or 40 and 140 nm; or 40 and 135 nm; or 40 and 130 nm; or 45 and 1,000 nm; or 45 and 500 nm; or 45 and 300 nm; or 45 and 200 nm; or 45 and 185 nm, or 45 and 175 nm; or 45 and 170 nm; or 45 and 165 nm; or 45 and 160 nm; or 45 and 155 nm; or 45 and 150 nm; or 45 and 145 nm; or 45 and 140 nm; or 45 and 135 nm; or 45 and 130 nm; or 50 and 1,000 nm; or 50 and 500 nm; or 50 and 300 nm; or 50 and 200 nm; or 50 and 185 nm, or 50 and 175 nm; or 50 and 170 nm; or 50 and 165 nm; or 50 and 160 nm; or 50 and 155 nm; or 50 and 150 nm; or 50 and 145 nm; or 50 and 140 nm; or 50 and 135 nm; or 50 and 130 nm; or 55 and 1,000 nm; or 55 and 500 nm; or 55 and 300 nm; or 55 and 200 nm; or 55 and 185 nm, or 55 and 175 nm; or 55 and 170 nm; or 55 and 165 nm; or 55 and 160 nm; or 55 and 155 nm; or 55 and 150 nm; or 55 and 145 nm; or 55 and 140 nm; or 55 and 135 nm; or 55 and 130 nm; or 60 and 1,000 nm; or 60 and 500 nm; or 60 and 300 nm; or 60 and 200 nm; or 60 and 185 nm, or 60 and 175 nm; or 60 and 170 nm; or 60 and 165 nm; or 60 and 160 nm; or 60 and 155 nm; or 60 and 150 nm; or 60 and 145 nm; or 60 and 140 nm; or 60 and 135 nm; or 60 and 130 nm; or 65 and 1,000 nm; or 65 and 500 nm; or 65 and 300 nm; or 65 and 200 nm; or 65 and 185 nm, or 65 and 175 nm; or 65 and 170 nm; or 65 and 165 nm; or 65 and 160 nm; or 65 and 155 nm; or 65 and 150 nm; or 65 and 145 nm; or 65 and 140 nm; or 65 and 135 nm; or 65 and 130 nm; or 70 and 1,000 nm; or 70 and 500 nm; or 70 and 300 nm; or 70 and 200 nm; or 70 and 185 nm, or 70 and 175 nm; or 70 and 170 nm; or 70 and 165 nm; or 70 and 160 nm; or 70 and 155 nm; or 70 and 150 nm; or 70 and 145 nm; or 70 and 140 nm; or 70 and 135 nm; or 70 and 130 nm; or 75 and 1,000 nm; or 75 and 500 nm; or 75 and 300 nm; or 75 and 200 nm; or 75 and 185 nm, or 75 and 175 nm; or 75 and 170 nm; or 75 and 165 nm; or 75 and 160 nm; or 75 and 155 nm; or 75 and 150 nm; or 75 and 145 nm; or 75 and 140 nm; or 75 and 135 nm; or 75 and 130 nm; or 90 and 1,000 nm; or 90 and 500 nm; or 90 and 300 nm; or 90 and 200 nm; or 90 and 185 nm, or 90 and 175 nm; or 90 and 170 nm; or 90 and 165 nm; or 90 and 160 nm; or 90 and 155 nm; or 90 and 150 nm; or 90 and 145 nm; or 90 and 140 nm; or 90 and 135 nm; or 90 and 130 nm; or 95 and 1,000 nm; or 95 and 500 nm; or 95 and 300 nm; or 95 and 200 nm; or 95 and 185 nm, or 95 and 175 nm; or 95 and 170 nm; or 95 and 165 nm; or 95 and 160 nm; or 95 and 155 nm; or 95 and 150 nm; or 95 and 145 nm; or 95 and 140 nm; or 95 and 135 nm; or 95 and 130 nm; or 100 and 1,000 nm; or 100 and 500 nm; or 100 and 300 nm; or 100 and 200 nm; or 100 and 185 nm, or 100 and 175 nm; or 100 and 170 nm; or 100 and 165 nm; or 100 and 150 nm; or 100 and 155 nm; or 100 and 150 nm; or 100 and 145 nm; or 100 and 100 nm; or 100 and 135 nm; or 100 and 130 nm; or 105 and 1,000 nm; or 105 and 500 nm; or 105 and 300 nm; or 105 and 200 nm; or 105 and 185 nm, or 105 and 175 nm; or 105 and 170 nm; or 105 and 165 nm; or 105 and 160 nm; or 105 and 155 nm; or 105 and 150 nm; or 105 and 145 nm; or 105 and 140 nm; or 105 and 135 nm; or 105 and 130 nm; or 110 and 1,000 nm; or 110 and 500 nm; or 110 and 300 nm; or 110 and 200 nm; or 110 and 185 nm, or 110 and 175 nm; or 110 and 170 nm; or 110 and 165 nm; or 110 and 150 nm; or 110 and 155 nm; or 110 and 150 nm; or 110 and 145 nm; or 110 and 100 nm; or 110 and 135 nm; or 110 and 130 nm; or 115 and 1,000 nm; or 115 and 500 nm; or 115 and 300 nm; or 115 and 200 nm; or 115 and 185 nm, or 115 and 175 nm; or 115 and 170 nm; or 115 and 165 nm; or 115 and 160 nm; or 115 and 155 nm; or 115 and 150 nm; or 115 and 145 nm; or 115 and 140 nm; or 115 and 135 nm; or 115 and 130 nm; or 120 and 1,000 nm; or 120 and 500 nm; or 120 and 300 nm; or 120 and 200 nm; or 120 and 185 nm, or 120 and 175 nm; or 120 and 170 nm; or 120 and 165 nm; or 120 and 150 nm; or 120 and 155 nm; or 120 and 150 nm; or 120 and 145 nm; or 120 and 100 nm; or 120 and 135 nm; or 120 and 130 nm.
48. The method of any of the preceding embodiments, wherein the average particle size of the extracellular vesicles in said composition is between 50 and 500 nm.
49. The method of any of the preceding embodiments, wherein the average particle size of the extracellular vesicles in said composition is between 100 and 200 nm.
50. The method of any of the preceding embodiments, wherein the average particle size of the extracellular vesicles in said composition is between 100 and 200 nm, and wherein 95% of the extracellular vesicles have a particle size between 100 and 200 nm.
51. The method of any of the preceding embodiments, wherein the average particle size of the extracellular vesicles in said composition is about 150 nm.
52. The method of any of the preceding embodiments, wherein the average particle size of the extracellular vesicles in said composition is about 150 nm, and wherein 95% of the extracellular vesicles have a particle size between 100 and 200 nm.
53. The method of any of the preceding embodiments wherein the extracellular vesicles are milk extracellular vesicles.
54. The method of any of the preceding embodiments wherein the extracellular vesicles are bovine milk extracellular vesicles.
55. The method of any of the preceding embodiments wherein the extracellular vesicles are bovine milk extracellular vesicles; and wherein the bovine milk extracellular vesicles comprise bovine IgG.
56. The method of any of the preceding embodiments, wherein the extracellular vesicles are milk extracellular vesicles.
57. The method of any one of the preceding embodiments, wherein the extracellular vesicles are milk extracellular vesicles, and wherein the extracellular vesicles are isolated from milk using a method of any of the “Isolation of Extracellular Vesicles From Milk” embodiments.
58. The method of any of the preceding embodiments wherein the extracellular vesicles comprise one or more cargo molecules.
59. The method of any of the preceding embodiments wherein the composition is a pharmaceutically acceptable composition.
60. The method of any of the preceding embodiments, wherein the Tryptophan or Tryptophan analog consists of one or more from the list including L-Tryptophan, D-Tryptophan, Tryptophan, Tryptophan dimers, Tryptophan Trimers, Tryptophan peptides, Histidine, L-Histidine, D-Histidine, Histidine Dimers, Histidine Trimers, Histidine peptides, Tyrosine, L-Tyrosine, D-Tyrosine, Tyrosine Dimers, Tyrosine Trimers, Tyrosine peptides, Arginine, L-Arginine, D-Arginine, Arginine Dimers, Arginine Trimers, Arginine peptides, Cysteine, L-Cysteine, D-Cysteine, Cysteine Dimers, Cysteine Trimers, Cysteine peptides, Lysine, L-Lysine, D-Lysine, Lysine Dimers, Lysine Trimers, Lysine Peptides, Phenylalanine, L-Phenylalanine, D-Phenylalanine, Phenylalanine Dimers, Phenylalanine Trimers, Phenylalanine peptides D-Carnitine, L-Carnitine, Carnitine dimers, Carnitine trimers, Carnitine peptides, Acetyl L-Carnitine, L-Carnitine L-tartrate, Propionyl-L-carnitine, D-Taurine, L-Taurine, Taurine dimers, Taurine trimers, Taurine peptides

### Lyophilization of Milk Extracellular Vesicles Embodiments

1. A composition comprising extracellular vesicles wherein said composition comprises above 0.01 μM Tryptophan or Tryptophan Analog.
2. A composition comprising extracellular vesicles wherein said composition comprises above 0.001% W/W % Tryptophan or Tryptophan analog.
3. A composition comprising extracellular vesicles wherein said composition comprises between 0.01 μM and 100 mM Tryptophan or Tryptophan analog.
4. A composition comprising extracellular vesicles wherein said composition comprises between 0.001% W/W % and 90% W/W % Tryptophan or Tryptophan analog.
5. A composition comprising extracellular vesicles wherein said composition comprises between 0.1 μM and 10 mM Tryptophan or Tryptophan analog.
6. A composition comprising extracellular vesicles wherein said composition comprises between 0.01% W/W % and 50% W/W % Tryptophan or Tryptophan analog.
7. A composition comprising extracellular vesicles wherein said composition comprises between 1 μM and 1 mM Tryptophan or Tryptophan analog.
8. A composition comprising extracellular vesicles wherein said composition comprises between 0.1% W/W % and 10% W/W % Tryptophan or Tryptophan analog.
9. A composition comprising extracellular vesicles wherein said composition comprises between 10 μM and 100 μM Tryptophan or Tryptophan analog.
10. A composition comprising extracellular vesicles wherein said composition comprises between 0.2% W/W % and 1% W/W % Tryptophan or Tryptophan analog.
11. A composition comprising milk extracellular vesicles wherein said composition comprises above 0.01 μM Tryptophan or Tryptophan Analog.
12. A composition comprising milk extracellular vesicles wherein said composition comprises above 0.001% W/W % Tryptophan or Tryptophan analog.
13. A composition comprising milk extracellular vesicles wherein said composition comprises between 0.01 μM and 100 mM Tryptophan or Tryptophan analog.
14. A composition comprising milk extracellular vesicles wherein said composition comprises between 0.001% W/W % and 90% W/W % Tryptophan or Tryptophan analog.
15. A composition comprising milk extracellular vesicles wherein said composition comprises between 0.1 μM and 10 mM Tryptophan or Tryptophan analog.
16. A composition comprising milk extracellular vesicles wherein said composition comprises between 0.01% W/W % and 50% W/W % Tryptophan or Tryptophan analog.
17. A composition comprising milk extracellular vesicles wherein said composition comprises between 1 μM and 1 mM Tryptophan or Tryptophan analog.
18. A composition comprising milk extracellular vesicles wherein said composition comprises between 0.1% W/W % and 10% W/W % Tryptophan or Tryptophan analog.
19. A composition comprising milk extracellular vesicles wherein said composition comprises between 10 μM and 100 μM Tryptophan or Tryptophan analog.
20. A composition comprising milk extracellular vesicles wherein said composition comprises between 0.2% W/W % and 1% W/W % Tryptophan or Tryptophan analog.
21. A composition comprising bovine milk extracellular vesicles wherein said composition comprises above 0.01 μM Tryptophan or Tryptophan Analog.
22. A composition comprising bovine milk extracellular vesicles wherein said composition comprises above 0.001% W/W % Tryptophan or Tryptophan analog.
23. A composition comprising bovine milk extracellular vesicles wherein said composition comprises between 0.01 μM and 100 mM Tryptophan or Tryptophan analog.
24. A composition comprising bovine milk extracellular vesicles wherein said composition comprises between 0.001% W/W % and 90% W/W % Tryptophan or Tryptophan analog.
25. A composition comprising bovine milk extracellular vesicles wherein said composition comprises between 0.1 μM and 10 mM Tryptophan or Tryptophan analog.
26. A composition comprising bovine milk extracellular vesicles wherein said composition comprises between 0.01% W/W % and 50% W/W % Tryptophan or Tryptophan analog.
27. A composition comprising bovine milk extracellular vesicles wherein said composition comprises between 1 μM and 1 mM Tryptophan or Tryptophan analog.
28. A composition comprising bovine milk extracellular vesicles wherein said composition comprises between 0.1% W/W % and 10% W/W % Tryptophan or Tryptophan analog.
29. A composition comprising bovine milk extracellular vesicles wherein said composition comprises between 10 μM and 100 μM Tryptophan or Tryptophan analog.
30. A composition comprising bovine milk extracellular vesicles wherein said composition comprises between 0.2% W/W % and 1% W/W % Tryptophan or Tryptophan analog.
31. The composition of any of the preceding embodiments, wherein said composition comprises between 0.1 μM and 1 mM; or 1.0 μM and 750 μM; or 10 μM and 500 μM; or 20 μM and 300 μM; or 40 μM and 200 μM; or 50 μM and 150 μM Tryptophan or a Tryptophan analog.
32. The composition of any of the preceding embodiments, wherein said composition comprises about 100 μM Tryptophan or a Tryptophan analog.
33. A composition comprising extracellular vesicles wherein said extracellular vesicles comprise above 0.01 μM Tryptophan or Tryptophan Analog.
34. A composition comprising extracellular vesicles wherein said extracellular vesicles comprise above 0.001% W/W % Tryptophan or Tryptophan analog.
35. A composition comprising extracellular vesicles wherein said extracellular vesicles comprise between 0.01 μM and 100 mM Tryptophan or Tryptophan analog.
36. A composition comprising extracellular vesicles wherein said extracellular vesicles comprise between 0.001% W/W % and 90% W/W % Tryptophan or Tryptophan analog.
37. A composition comprising extracellular vesicles wherein said extracellular vesicles comprise between 0.1 μM and 10 mM Tryptophan or Tryptophan analog.
38. A composition comprising extracellular vesicles wherein said extracellular vesicles comprise between 0.01% W/W % and 50% W/W % Tryptophan or Tryptophan analog.
39. A composition comprising extracellular vesicles wherein said extracellular vesicles comprise between 1 μM and 1 mM Tryptophan or Tryptophan analog.
40. A composition comprising extracellular vesicles wherein said extracellular vesicles comprise between 0.1% W/W % and 10% W/W % Tryptophan or Tryptophan analog.
41. A composition comprising extracellular vesicles wherein said extracellular vesicles comprise between 10 μM and 100 μM Tryptophan or Tryptophan analog.
42. A composition comprising extracellular vesicles wherein said extracellular vesicles comprise between 0.2% W/W % and 1% W/W % Tryptophan or Tryptophan analog.
43. A composition comprising milk extracellular vesicles wherein said extracellular vesicles comprise above 0.01 μM Tryptophan or Tryptophan Analog.
44. A composition comprising milk extracellular vesicles wherein said extracellular vesicles comprise above 0.001% W/W % Tryptophan or Tryptophan analog.
45. A composition comprising milk extracellular vesicles wherein said extracellular vesicles comprise between 0.01 μM and 100 mM Tryptophan or Tryptophan analog.
46. A composition comprising milk extracellular vesicles wherein said extracellular vesicles comprise between 0.001% W/W % and 90% W/W % Tryptophan or Tryptophan analog.
47. A composition comprising milk extracellular vesicles wherein said extracellular vesicles comprise between 0.1 μM and 10 mM Tryptophan or Tryptophan analog.
48. A composition comprising milk extracellular vesicles wherein said extracellular vesicles comprise between 0.01% W/W % and 50% W/W % Tryptophan or Tryptophan analog.
49. A composition comprising milk extracellular vesicles wherein said extracellular vesicles comprise between 1 μM and 1 mM Tryptophan or Tryptophan analog.
50. A composition comprising milk extracellular vesicles wherein said extracellular vesicles comprise between 0.1% W/W % and 10% W/W % Tryptophan or Tryptophan analog. 51. A composition comprising milk extracellular vesicles wherein said extracellular vesicles comprise between 10 μM and 100 μM Tryptophan or Tryptophan analog.
52. A composition comprising milk extracellular vesicles wherein said extracellular vesicles comprise between 0.2% W/W % and 1% W/W % Tryptophan or Tryptophan analog.
53. A composition comprising bovine milk extracellular vesicles wherein said extracellular vesicles comprise above 0.01 μM Tryptophan or Tryptophan Analog.
54. A composition comprising bovine milk extracellular vesicles wherein said extracellular vesicles comprise above 0.001% W/W % Tryptophan or Tryptophan analog.
55. A composition comprising bovine milk extracellular vesicles wherein said extracellular vesicles comprise between 0.01 μM and 100 mM Tryptophan or Tryptophan analog.
56. A composition comprising bovine milk extracellular vesicles wherein said extracellular vesicles comprise between 0.001% W/W % and 90% W/W % Tryptophan or Tryptophan analog.
57. A composition comprising bovine milk extracellular vesicles wherein said extracellular vesicles comprise between 0.1 μM and 10 mM Tryptophan or Tryptophan analog.
58. A composition comprising bovine milk extracellular vesicles wherein said extracellular vesicles comprise between 0.01% W/W % and 50% W/W % Tryptophan or Tryptophan analog.
59. A composition comprising bovine milk extracellular vesicles wherein said extracellular vesicles comprise between 1 μM and 1 mM Tryptophan or Tryptophan analog.
60. A composition comprising bovine milk extracellular vesicles wherein said extracellular vesicles comprise between 0.1% W/W % and 10% W/W % Tryptophan or Tryptophan analog.
61. A composition comprising bovine milk extracellular vesicles wherein said extracellular vesicles comprise between 10 μM and 100 μM Tryptophan or Tryptophan analog.
62. A composition comprising bovine milk extracellular vesicles wherein said extracellular vesicles comprise between 0.2% W/W % and 1% W/W % Tryptophan or Tryptophan analog.
63. The composition of any of the preceding embodiments, wherein said extracellular vesicles comprise between 0.1 μM and 1 mM; or 1.0 μM and 750 μM; or 10 M and 500 μM; or 20 μM and 300 μM; or 40 μM and 200 μM; or 50 μM and 150 μM Tryptophan or a Tryptophan analog.
64. The composition of any of the preceding embodiments, wherein said extracellular vesicles comprise about 100 μM Tryptophan or a Tryptophan analog.
65. The composition of any of the preceding embodiments, wherein said Tryptophan or Tryptophan analog comprises one or more selected from the group consisting of L-Tryptophan, D-Tryptophan, Tryptophan, Tryptophan dimers, Tryptophan Trimerstrimers, Tryptophan peptides, Histidine, L-Histidine, D-Histidine, Histidine Dimersdimers, Histidine Trimerstrimers, Histidine peptides, Tyrosine, L-Tyrosine, D-Tyrosine, Tyrosine Dimersdimers, Tyrosine Trimerstrimers, Tyrosine peptides, Arginine, L-Arginine, D-Arginine, Arginine Dimersdimers, Arginine Trimerstrimers, Arginine peptides, Cysteine, L-Cysteine, D-Cysteine, Cysteine Dimersdimers, Cysteine Trimerstrimers, Cysteine peptides, Lysine, L-Lysine, D-Lysine, Lysine Dimersdimers, Lysine Trimerstrimers, Lysine Peptides, Phenylalanine, L-Phenylalanine, D-Phenylalanine, Phenylalanine Dimersdimers, Phenylalanine Trimerstrimers, Phenylalanine peptides, D-Carnitine, L-Carnitine, Carnitine dimers, Carnitine trimers, Carnitine peptides, Acetyl L-Carnitine, L-Carnitine L-tartrate, Propionyl-L-carnitine, D-Taurine, L-Taurine, Taurine dimers, Taurine trimers, Taurine peptides
66. The composition of any of the preceding embodiments, wherein the average particle size of the extracellular vesicles in said composition are between 10 and 1000 nm.
67. The composition of any of the preceding embodiments, wherein the average particle size of the extracellular vesicles in said composition is between 10 and 1000 nm, and wherein at least 75%, or 80%; or 90%, or 95%, or 98%, or 99% of the particles are between 10 and 1000 nm. 68. The composition of any of the preceding embodiments, wherein at least 75%, or 80%; or 90% or 95% of the particles in said composition are less than 600 nm.
69. The composition of any of the preceding embodiments, wherein at least 95% of the particles in said composition are less than 600 nm.
70. The composition of any of the preceding embodiments, wherein at least 99% of the particles in said composition are less than 600 nm.
71. The composition of any of the preceding embodiments, wherein at least 75%, or 80%; or 90%, or 95%, or 98%, or 99% of the particles in said composition are less than about 500 nm.
72. The composition of any of the preceding embodiments, wherein at least 95% of the particles in said composition are less than about 500 nm.
73. The composition of any of the preceding embodiments, wherein at least 99% of the particles in said composition are less than about 500 nm.
74. The composition of any of the preceding embodiments, wherein at least 75%, or 80%; or 90%, or 95%, or 98%, or 99% of the particles in said composition are less than about 250 nm.
75. The composition of any of the preceding embodiments, wherein at least 95% of the particles in said composition are less than about 250 nm.
76. The composition of any of the preceding embodiments, wherein at least 99% of the particles in said composition are less than about 250 nm.
77. The composition of any of the preceding embodiments, wherein at least 75%, or 80%; or 90%, or 95%, or 98%, or 99% of the particles in said composition are greater than 20 nm.
78. The composition of any of the preceding embodiments, wherein at least 95% of the particles in said composition are greater than 20 nm.
79. The composition of any of the preceding embodiments, wherein at least 99% of the particles in said composition are greater than 20 nm.
80. The composition of any of the preceding embodiments, wherein the average particle size of the extracellular vesicles in said composition is between 10 and 500 nm; or 10 and 300 nm; or 10 and 200 nm; or 10 and 185 nm, or 10 and 175 nm; or 10 and 170 nm; or 10 and 165 nm; or 10 and 160 nm; or 10 and 155 nm; or 10 and 150 nm; or 10 and 145 nm; or 10 and 140 nm; or 10 and 135 nm; or 10 and 130 nm; or 15 and 1,000 nm; or 15 and 500 nm; or 15 and 300 nm; or 15 and 200 nm; or 15 and 185 nm, or 15 and 175 nm; or 15 and 170 nm; or 15 and 165 nm; or 15 and 160 nm; or 15 and 155 nm; or 15 and 150 nm; or 15 and 145 nm; or 15 and 140 nm; or 15 and 135 nm; or 15 and 130 nm; or 20 and 1,000 nm; or 20 and 500 nm; or 20 and 300 nm; or 20 and 200 nm; or 20 and 185 nm, or 20 and 175 nm; or 20 and 170 nm; or 20 and 165 nm; or 20 and 160 nm; or 20 and 155 nm; or 20 and 150 nm; or 20 and 145 nm; or 20 and 140 nm; or 20 and 135 nm; or 20 and 130 nm; or 25 and 1,000 nm; or 25 and 500 nm; or 25 and 300 nm; or 25 and 200 nm; or 25 and 185 nm, or 25 and 175 nm; or 25 and 170 nm; or 25 and 165 nm; or 25 and 160 nm; or 25 and 155 nm; or 25 and 150 nm; or 25 and 145 nm; or 25 and 140 nm; or 25 and 135 nm; or 25 and 130 nm; or 30 and 1,000 nm; or 30 and 500 nm; or 30 and 300 nm; or 30 and 200 nm; or 30 and 185 nm, or 30 and 175 nm; or 30 and 170 nm; or 30 and 165 nm; or 30 and 160 nm; or 30 and 155 nm; or 30 and 150 nm; or 30 and 145 nm; or 30 and 140 nm; or 30 and 135 nm; or 30 and 130 nm; or 35 and 1,000 nm; or 35 and 500 nm; or 35 and 300 nm; or 35 and 200 nm; or 35 and 185 nm, or 35 and 175 nm; or 35 and 170 nm; or 35 and 165 nm; or 35 and 160 nm; or 35 and 155 nm; or 35 and 150 nm; or 35 and 145 nm; or 35 and 140 nm; or 35 and 135 nm; or 35 and 130 nm; or 40 and 1,000 nm; or 40 and 500 nm; or 40 and 300 nm; or 40 and 200 nm; or 40 and 185 nm, or 40 and 175 nm; or 40 and 170 nm; or 40 and 165 nm; or 40 and 160 nm; or 40 and 155 nm; or 40 and 150 nm; or 40 and 145 nm; or 40 and 140 nm; or 40 and 135 nm; or 40 and 130 nm; or 45 and 1,000 nm; or 45 and 500 nm; or 45 and 300 nm; or 45 and 200 nm; or 45 and 185 nm, or 45 and 175 nm; or 45 and 170 nm; or 45 and 165 nm; or 45 and 160 nm; or 45 and 155 nm; or 45 and 150 nm; or 45 and 145 nm; or 45 and 140 nm; or 45 and 135 nm; or 45 and 130 nm; or 50 and 1,000 nm; or 50 and 500 nm; or 50 and 300 nm; or 50 and 200 nm; or 50 and 185 nm, or 50 and 175 nm; or 50 and 170 nm; or 50 and 165 nm; or 50 and 160 nm; or 50 and 155 nm; or 50 and 150 nm; or 50 and 145 nm; or 50 and 140 nm; or 50 and 135 nm; or 50 and 130 nm; or 55 and 1,000 nm; or 55 and 500 nm; or 55 and 300 nm; or 55 and 200 nm; or 55 and 185 nm, or 55 and 175 nm; or 55 and 170 nm; or 55 and 165 nm; or 55 and 160 nm; or 55 and 155 nm; or 55 and 150 nm; or 55 and 145 nm; or 55 and 140 nm; or 55 and 135 nm; or 55 and 130 nm; or 60 and 1,000 nm; or 60 and 500 nm; or 60 and 300 nm; or 60 and 200 nm; or 60 and 185 nm, or 60 and 175 nm; or 60 and 170 nm; or 60 and 165 nm; or 60 and 160 nm; or 60 and 155 nm; or 60 and 150 nm; or 60 and 145 nm; or 60 and 140 nm; or 60 and 135 nm; or 60 and 130 nm; or 65 and 1,000 nm; or 65 and 500 nm; or 65 and 300 nm; or 65 and 200 nm; or 65 and 185 nm, or 65 and 175 nm; or 65 and 170 nm; or 65 and 165 nm; or 65 and 160 nm; or 65 and 155 nm; or 65 and 150 nm; or 65 and 145 nm; or 65 and 140 nm; or 65 and 135 nm; or 65 and 130 nm; or 70 and 1,000 nm; or 70 and 500 nm; or 70 and 300 nm; or 70 and 200 nm; or 70 and 185 nm, or 70 and 175 nm; or 70 and 170 nm; or 70 and 165 nm; or 70 and 160 nm; or 70 and 155 nm; or 70 and 150 nm; or 70 and 145 nm; or 70 and 140 nm; or 70 and 135 nm; or 70 and 130 nm; or 75 and 1,000 nm; or 75 and 500 nm; or 75 and 300 nm; or 75 and 200 nm; or 75 and 185 nm, or 75 and 175 nm; or 75 and 170 nm; or 75 and 165 nm; or 75 and 160 nm; or 75 and 155 nm; or 75 and 150 nm; or 75 and 145 nm; or 75 and 140 nm; or 75 and 135 nm; or 75 and 130 nm; or 90 and 1,000 nm; or 90 and 500 nm; or 90 and 300 nm; or 90 and 200 nm; or 90 and 185 nm, or 90 and 175 nm; or 90 and 170 nm; or 90 and 165 nm; or 90 and 160 nm; or 90 and 155 nm; or 90 and 150 nm; or 90 and 145 nm; or 90 and 140 nm; or 90 and 135 nm; or 90 and 130 nm; or 95 and 1,000 nm; or 95 and 500 nm; or 95 and 300 nm; or 95 and 200 nm; or 95 and 185 nm, or 95 and 175 nm; or 95 and 170 nm; or 95 and 165 nm; or 95 and 160 nm; or 95 and 155 nm; or 95 and 150 nm; or 95 and 145 nm; or 95 and 140 nm; or 95 and 135 nm; or 95 and 130 nm; or 100 and 1,000 nm; or 100 and 500 nm; or 100 and 300 nm; or 100 and 200 nm; or 100 and 185 nm, or 100 and 175 nm; or 100 and 170 nm; or 100 and 165 nm; or 100 and 150 nm; or 100 and 155 nm; or 100 and 150 nm; or 100 and 145 nm; or 100 and 100 nm; or 100 and 135 nm; or 100 and 130 nm; or 105 and 1,000 nm; or 105 and 500 nm; or 105 and 300 nm; or 105 and 200 nm; or 105 and 185 nm, or 105 and 175 nm; or 105 and 170 nm; or 105 and 165 nm; or 105 and 160 nm; or 105 and 155 nm; or 105 and 150 nm; or 105 and 145 nm; or 105 and 140 nm; or 105 and 135 nm; or 105 and 130 nm; or 110 and 1,000 nm; or 110 and 500 nm; or 110 and 300 nm; or 110 and 200 nm; or 110 and 185 nm, or 110 and 175 nm; or 110 and 170 nm; or 110 and 165 nm; or 110 and 150 nm; or 110 and 155 nm; or 110 and 150 nm; or 110 and 145 nm; or 110 and 100 nm; or 110 and 135 nm; or 110 and 130 nm; or 115 and 1,000 nm; or 115 and 500 nm; or 115 and 300 nm; or 115 and 200 nm; or 115 and 185 nm, or 115 and 175 nm; or 115 and 170 nm; or 115 and 165 nm; or 115 and 160 nm; or 115 and 155 nm; or 115 and 150 nm; or 115 and 145 nm; or 115 and 140 nm; or 115 and 135 nm; or 115 and 130 nm; or 120 and 1,000 nm; or 120 and 500 nm; or 120 and 300 nm; or 120 and 200 nm; or 120 and 185 nm, or 120 and 175 nm; or 120 and 170 nm; or 120 and 165 nm; or 120 and 150 nm; or 120 and 155 nm; or 120 and 150 nm; or 120 and 145 nm; or 120 and 100 nm; or 120 and 135 nm; or 120 and 130 nm.
81. The composition of any of the preceding embodiments, wherein the average particle size of the extracellular vesicles in said composition is between 50 and 500 nm.
82. The composition of any of the preceding embodiments, wherein the average particle size of the extracellular vesicles in said composition is between 100 and 200 nm.
83. The composition of any of the preceding embodiments, wherein the average particle size of the extracellular vesicles in said composition is between 100 and 200 nm, and wherein 95% of the extracellular vesicles have a particle size between 100 and 200 nm.
84. The composition of any of the preceding embodiments, wherein the average particle size of the extracellular vesicles in said composition is about 150 nm.
85. The composition of any of the preceding embodiments, wherein the average particle size of the extracellular vesicles in said composition is about 150 nm, and wherein 95% of the extracellular vesicles have a particle size between 100 and 200 nm.
86. The composition of any of the preceding embodiments wherein the extracellular vesicles in said composition are milk extracellular vesicles.
87. The composition of any of the preceding embodiments wherein the vesicles in said composition are bovine milk extracellular vesicles.
88. The composition of any of the preceding embodiments wherein the extracellular vesicles in said composition are bovine milk extracellular vesicles; and wherein the bovine milk extracellular vesicles comprise bovine IgG.
89. The composition of any of the preceding embodiments, wherein the extracellular vesicles in said composition are milk extracellular vesicles.
90. The composition of any one of the preceding embodiments, wherein the extracellular vesicles in said composition are milk extracellular vesicles, wherein the extracellular vesicles are isolated.
91. The composition of any one of the preceding embodiments, wherein the extracellular vesicles are milk extracellular vesicles, and wherein the extracellular vesicles are isolated from milk using a method of any of the “Isolation of Extracellular Vesicles From Milk” embodiments.
92. The composition of any of the preceding embodiments wherein the extracellular vesicles comprise one or more cargo molecules.
93. The composition of any of the preceding embodiments wherein the composition is a pharmaceutically acceptable composition.
94. The composition of any of the preceding embodiments wherein the composition also comprises Trehalose or a Trehalose analog at about 50 mM concentration.
95. The composition of any of the preceding embodiments wherein the Trehalose or Trehalose analog consists of one or more from the list including Trehalose, Sucrose, Lactose, Glucose, Disaccharide molecules.
96. The composition of any of the preceding embodiments wherein the composition is then frozen and lyophilized, or dried.
97. A method of lyophilizing drying bovine milk extracellular vesicles supplemented with Trehalose and Tryptophan.
98. A composition of lyophilized dried milk extracellular vesicles loaded with cargo molecules and supplemented with Trehalose or a Trehalose analog in addition to Tryptophan or a Tryptophan analog.
99. A method of contacting extracellular vesicles with Trehalose and Tryptophan wherein said extracellular vesicles are dried and/or frozen and lyophilized.
100. A method of contacting extracellular vesicles with Trehalose and Tryptophan wherein said extracellular vesicles are frozen and lyophilized.
101. A composition of extracellular vesicles loaded with cargo molecules listed herein and supplemented with Trehalose or a Trehalose analog in addition to Tryptophan or a Tryptophan analog.

### EXAMPLES

The present disclosure will be further described in the following examples, which do not limit the scope of the present disclosure.

### Example 1: Isolation of Extracellular Vesicles From Milk Using Tryptophan

Raw bovine milk was collected at 4C and defatted under industry standard conditions (centrifuged at 5,000× g to remove fat), resulting in skim milk. Half of the skim milk was treated with an FDA approved method of pasteurization. The two batches of skim milk were then subjected to 2.5 μm filtration and 0.8 μm filtration, then the permeate was treated with 150 μM tryptophan for 10 minutes on ice, while the retenate was discarded. The solution was then filtered through 0.45 μm and 0.22 μm filters, with the retenate being discarded following each filtration. The solution was then filtered on a 500 kDa filter until the retenate volume was 20% of the starting volume, and this solution was stored. This sample was then separated using a 70 nm sepharose column. Fractions 7-9 were collected and pooled, representing a purified milk extracellular vesicle population.

This solution was then used for in depth analysis and experimentation including a number of standard and unique assays. For example, the protein was analyzed using Nanodrop for protein quantification, and Western blotting for protein markers. Protein is also analyzed using single molecule localization microscopy for protein markers and subpopulation analysis, but also gain insights into size and concentration using this approach; and SMLM approaches were validated using Nanoparticle Tracking Analysis for size and concentration investigation. We also utilize Zetasizer measurement for zeta potential, Transmission Electron Microscopy for visualization, confocal microscopy for investigation of vesicle patency, and in vitro bioactivity measurements of vesicles using an automated electrical wounding protocol (ECIS).

ECIS is “Electric Cell-Substrate Impedance Sensing”-protocol used provided: The cellular monolayer's confluence was measured in real time with an electric cell-substrate impedance sensing (ECIS) instrument (Applied Biophysics, NY, USA) to monitor their migratory response. Briefly, 8W1E or 96WIE+electrode ECIS arrays were coated with 10 mM L-cysteine, followed by 1% gelatin according to manufacturer guidelines. Electrodes were then stabilized in HuDF complete media, and subsequently seeded with HuDF cells at a density of 1×10⁵ cells/well. Cells were incubated for 8-10 hours to achieve at least 80% confluency, which was confirmed by previously performed growth-phase profiles. 8 hours before wound induction, media was changed to complete media supplemented with 10 μg/mL mitomycin C to limit cell proliferation. A 25 kHz, 2600 μA, 20 second electric wound was then applied for cells and impedance was monitored until cell migration was complete. Cell migration rate was determined by a custom Python script that tracked changes in impedance over time. The impedance of cell-covered electrodes was measured with the multiple frequencies over time (MFT) mode at eleven frequencies between 62.5 to 64000 Hz (62.5, 125, 250, 500, 1000, 2000, 4000, 8000,16000, 32000, 64000). Cell monolayers were treated with mEVs at optimized concentrations determined via UV-Vis intensity at 260/280nm. Each result was normalized to a control well present in each electrode array.

Nanodrop analysis was performed using the Nanodrop 2000 as per the manufacturer's instructions under 260/280 wavelength stimulation to quantify protein expression in the sample. Basic western blotting protocols are followed using antibodies including CD81, CD9, Calnexin, Syntenin-1, TSG-101, Pannexin-1, Connexin-43, Lactadherin and Casein. Single Molecule Localization Microscopy was performed using the EV profiler kit using procedures from the kits instructions, with antibodies including CD81, CD9, CD63, Pannexin-1, P2X7 and Connexin-43. Nanoparticle Tracking analysis was performed on a Nanosight NS 300 using a dilution of 1:10,000 in a standard buffer solution and analyzed using a 405 nm laser. Zetasizer measurements were performed on a Horiba Scientific SZ-100V2 nanoparticle analyzer using the manufacturer's instructions. Transmission electron microscopy was performed using formvar coated copper grids coated with 0.1% lysine. Samples were adhered to grids for 10 minutes then counterstained with uranlyess solution for 1 minute, then left overnight prior to imaging. Confocal microscopy was performed on milk vesicles loaded with Calcein AM dye on a Leica SP8. Vesicles were administered to standard microscope slides prior to coverslipping and imaging at 63× under 488 nm laser stimulation. In vitro bioactivity measurements were performed using an electric cell-substrate impedance sensing (ECIS) instrument from Applied Biophysics.

### Example 2: Isolation of Extracellular Vesicles From Milk Using Tryptophan and EDTA

Raw bovine milk was collected at 4C and defatted under industry standard conditions (centrifuged at 5,000× g to remove fat), resulting in skim milk. Skim milk was then subjected to 1.2 μm filtration, then the permeate was treated with 30 mM EDTA and 150 μM tryptophan for 30 minutes at room (ambient, 20 C) temperature, while the retenate was discarded. The solution was then filtered through 0.45 μm and 0.22 μm filters, with the retenate being discarded following each filtration. The solution was then filtered on a 500 kDa filter until the retenate volume was 20% of the starting volume, and this solution was stored. This sample was then separated using a 70 nm sepharose column. Fractions 7-9 were collected and pooled, representing a purified milk extracellular vesicle population. Optionally, this sample was then buffered with 50 mM trehalose and lyophilized as described in example 7. This solution was then used for in depth analysis and experimentation, as described in Example 1.

### Example 3: Isolation of Extracellular Vesicles From Milk Using Tryptophan and acid pH

Raw bovine milk was collected at 4C and defatted under industry standard conditions resulting in skim milk as in example 1. Skim milk was then subjected to 1.2 um filtration, then the permeate was treated with 150 μM tryptophan for 60 minutes at room (ambient, 20 °C) temperature, while the retenate was discarded. The permeate was then acidified to pH 4.6 by addition of HCL or citric acid, which was then removed as a pellet by centrifugation at 5,000× g, or alternatively may be removed by raising temperature to 45-50 degrees Celsius for 5 -15 minutes to form a hard curd precipitate, which may then be strained from the solution. The supernatant solution following pelleting at 5,000 × g was then filtered through 0.45 and 0.22 filters, with the retenate being discarded following each filtration. The solution was then filtered on a 500 kDa filter until the retenate volume was 20% of the starting volume, and this solution was stored. This sample was then separated using a 70 nm sepharose column. Fractions 7-9 were collected and pooled, representing a purified milk extracellular vesicle population. Optionally, this sample was then buffered with 50 mM Trehalose and lyophilized as described in example 7. This solution was then used for in depth analysis and experimentation, as described in Example 1.

### Example 4: Isolation of Extracellular Vesicles from Milk Using Tryptophan, Acid pH, and Temperature

Raw bovine milk was collected at 4C and defatted under industry standard conditions resulting in skim milk as in examples 1 and 3. Skim milk was then treated with 150 μM Tryptophan and was acidified by addition of citric acid, sulfuric acid, or hydrochloric acid and treated at 55 °C for 30 minutes to form a hard curd precipitate, which was then strained from the solution. The supernatant solution following curd formation was then filtered through 0.45 and 0.22 micron filters, with the retenate being discarded following each filtration, and the permeate being collected. The permeate solution was then filtered on a 500 kDa filter until the retenate volume was ~20% of the starting volume, and this solution was stored. This sample was then separated on a 70 nm sepharose column. Fractions 7-9 were collected and pooled, representing a purified milk extracellular vesicle population. Optionally, this sample was then buffered with 50 mM Trehalose and 100 μM Tryptophan and lyophilized as described in example 7. This solution was then used for in depth analysis and experimentation, as described in Example 1.

### Example 5: ATP-Milk Extracellular Vesicles

Milk vesicles, or extracellular vesicles, or exosomes as collected by examples described herein or as described previously (US patent application for isolation?) were collected and incubated with adenosine triphosphate (ATP) at concentrations including 100 nm, 500 nm, 1.0 M, 2.0 μM, 5.0 μM, 10 μM, and 50 μM for 30 minutes at 37 °C. Optimal concentrations of ATP for enhanced bioactivity were found at 2.0 μM and 1.0 μM, with final ATP concentrations in the milk vesicle composition ranging from 100 nM to 10 μM.

### Example 6: Assessment of ATP-Extracellular Vesicles

ATP-Extracellular Vesicle combinations were then analyzed using a number of methods. Bioactivity was first measured using an in vitro analysis procedure. Cells used were human dermal fibroblasts (HuDF; ATCC, PCS-201-012) as well as two lineages of Madin Darby Canine Kidney (MDCK) Cells-one being Cx43 enhanced (Cx43+), and the other being Cx43 reduced (Cx43−). Medium used for HuDF was DMEM HG (4.5 g/L Glucose) with 2% Normal Calf Serum (NCS; Thermo Fisher/Gibco, 16010-159) and 4% Fetal Bovine Serum (FBS; Thermo Fisher/Gibco, 26140-079). Medium used for the Cx 43+ MDCK cells was M199 (Millipore Sigma M 4530) supplemented with 10% FBS and 1% Hygromycin B (Sigma H0654), while Cx43− MDCKs used M199 with 10% FBS and 1:125 Sodium Pyruvate (Invitrogen 11360-070). Cells were expanded to confluency, then passaged onto sterile coverslips in a 12-well plate, then given 2 days to adhere and grow prior to assay. Assay was performed by using a sterile pipette tip to scratch the surface of the cells, then cells were rinsed 1× in dPBS (Invitrogen) and provided fresh culture medium supplemented with CTDR-tagged milk EVs. Cells were given 15 minutes to take up ATP-Vesicle combinations, then were rinsed in 1× dPBS and fixed in 2% Paraformaldehyde (Fisher O 4042-500). Cells were rinsed 4× in PBS, then stained in 1:20,000 Hoechst (Life Technologies, H3569) and rinsed one additional time in PBS. Coverslips were then removed and adhered to microscope slide, and imaged on a Leica SP8. Images were analyzed in ImageJ by saving the individual red channel and converting to 8-bit, thresholding, and counting particle numbers.

ATP-Extracellular Vesicles may also be analyzed by Mass-Spectrometry for ATP concentrations to validate the presence of ATP within the EVs. Samples can be prepared by using trichloroacetic acid/acetone steps from a 2-D clean up kit from Bio-Rad. Following protein precipitation, samples are resolubilized in 10 uL 8 M urea, 50 mM NH₄HCO₃, reduced and alkylated using dithiothritol and iodoacetamide, then digested for three hours at room temperature using endo-Lys-C. Samples are then diluted to 2M urea using 50 mM NH₄HCO₃, and digestion is continued overnight by the addition of sequencing-grade modified trypsin from Promega. Samples are then acidified by 2% formic acid, desalted on pre-equilibrated columns, and washed using an EASY nLC-1200 system. Column is maintained at constant temperature and interfaced online. Peptides may be eluted from the column using a binary gradient that steps from 5% solvent to 95% solvent over 60 mins. MS data is acquired by recording full scan spectra.

Extracellular Vesicle concentrations of ATP may be readily determined in a number of manners-there are a wide range of ATP concentration determination assays, with the following example provided as the basic mechanism of ATP detection in a solution. The first step is to administer firefly luciferase enzyme and substrate to activate luciferin with present ATP, which then gives luciferyl-adenylate and pyrophosphate. The following chemical reaction results in a photoelectric stimulation of oxyluciferin, which emits a green/yellow luminescent signal (550-570 nm). The solution luminescence is then read using a luminometer, and is sensitive to concentrations of ATP<50 pg/mL. Other methods utilize fluorescent capture, which enables longer term signal capture periods, and are additionally viable methods to detect and measure ATP concentration.

### Example 7: Lyophilization of Milk Extracellular Vesicles

Milk extracellular vesicles, or extracellular vesicles, or exosomes as collected by examples described herein or as described previously (for example as in WO2022182782) were collected and incubated in a number of chemical excipients. These include concentrations of 10 mM, 50 mM and 200 mM of Sucrose, lactose, Trehalose, TMAO and tryptophan, as well as mixtures thereof, prior to freezing, in order to result in a mixture with a glass transition temperature between 40 and 80 °C. Samples were frozen at −80 °C and subjected to shelf lyophilization at a condenser temperature of-100C under a vacuum pressure of 10 mTorr. Additionally, methods used for lyophilizing a composition of vesicles include A) freezing the composition by either cooling at a controlled rate of 1 ° C per minute to a frozen temperature, or; flash freezing the composition using liquid nitrogen, dry ice, or other flash freezing method. B) Primary Drying the composition by adjusting temperature and pressure according to the specific container and constitution of the composition, such adjustments being within the realm of common knowledge for those skilled in the art. C) Secondary Drying the composition by further reducing the moisture content of the composition by modulating temperature and vacuum conditions post-sublimation, and; Tailoring the secondary drying phase parameters, including temperature and pressure ramping, based on the specific properties of the container and the composition, such tailoring being obvious to those skilled in the art. D) Vacuum capping samples post-lyophilization. Dried samples were stored at room temperature or at 4 °C under vacuum or protected from moisture by desiccant. This solution was then used for in depth analysis and experimentation including a number of standard and unique assays. We first analyze protein using Nanodrop for protein quantification, then use Nanoparticle Tracking Analysis for size and concentration investigation. We also utilize Zetasizer measurement for zeta potential, Transmission Electron Microscopy for visualization, confocal microscopy for investigation of vesicle patency, and in vitro bioactivity measurements using an automated electrical wounding protocol. Optimized formulation for lyophilization protection of milk extracellular vesicles was found to be 50 mM Trehalose buffered with 100 μM Tryptophan, enabling 6+ month shelf stability of our formulations. LC-MS analysis of samples will elucidate the presence of Tryptophan, a novel excipient in the lyophilization of milk extracellular vesicles.

### Example 8: Storage Materials & Methods for Storage Stability Testing of Lyophilized Milk Extracellular Vesicles

Isolated milk extracellular vesicles, or extracellular vesicles, or exosomes loaded with act11 peptide using the protocols previously described were lyophilized in the presence of 50 mM Trehalose or Trehalose analog and 100 μM Tryptophan or Tryptophan analog and stored in a closed container with desiccant at room temperature. At given time points, samples were reconstituted using an equal volume of deionized H₂O to the original amount of mEV volume within the composition. Equivalent doses of each sample were added to a single ECIS cell with a final volume of 400 uL post-wound as previously described in section 83. At 0 months and 6 months, samples were analyzed via NTA and particles/mL were compared.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20 %, or up to ±10 %, or up to ±5 %, or up to ±1 % of a given value. Alternatively, particularly with respect to biological systems or processes, the term can mean within an order of magnitude, such as within 2-fold, of a value. For the avoidance of doubt, a value described herein with an “about” modifier specifically includes the stated value itself, for example if a value is expressed as “about 10”—the value of exactly 10 is specifically contemplated. As used herein, ranges can be expressed as from “about” one particular value, or “about” one value to “about” another particular value. It is also understood that each unit between two particular units are also disclosed. For example, if the range of “10-15” is disclosed, then 11, 12, 13, and 14 are also disclosed.

All references referred to in the present disclosure are hereby incorporated by reference in their entirety. Various embodiments of the present disclosure may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

The embodiments of the disclosure described above are intended to be merely exemplary; numerous variations and modifications will be apparent to those skilled in the art. All such variations and modifications are intended to be within the scope of the present disclosure as defined in any appended claims.

What is claimed is:

1. A composition comprising extracellular vesicles, wherein said extracellular vesicles comprise between 0.1 and 2 μM ATP or an ATP analogue.

2. The composition of claim 1, wherein said extracellular vesicles comprise between 0.5 and 1.5 μM ATP or an ATP analogue.

3. The composition of claim 2, wherein said extracellular vesicles comprise between 0.5 and 1.5 μM ATP.

4. The composition of claim 3, wherein said extracellular vesicles are milk extracellular vesicles.

5. The composition of claim 4, wherein said extracellular vesicles are bovine milk extracellular vesicles.

6. The composition of claim 5, wherein the average particle size of the extracellular vesicles is between 100 and 200 nm.

7. The composition of claim 6, wherein 99% of the extracellular vesicles have a particle size that is greater than 20 nm and less than 600 nm.

8. A method of preparing a composition of claim 7, wherein the method comprises contacting bovine milk extracellular vesicles with ATP.

9. The composition of claim 8, wherein said extracellular vesicles are loaded with one or more cargo molecules.

10. The composition of claim 9, wherein cargo molecules comprise one or more molecule types selected from the group consisting of a nucleic acid, a polypeptide, a carbohydrate and a steroid.

11. A method, comprising administering a composition of claim 10 to a subject.

12. The method of claim 11, wherein said composition is administered to said subject orally, intranasally, parenterally, or intravenously.

13. A method of isolating extracellular vesicles from a milk sample, wherein the method comprises (i) contacting the milk with tryptophan or a tryptophan analog at a concentration between 50 μM and 300 μM for between 10 and 60 minutes at a pH between 2.0 and 8.0 and a temperature between 10 degrees Celsius and 80 degrees Celsius, and (ii) separating said extracellular vesicles from other components of milk by filtration and/or centrifugation.

14. The method of claim 13, wherein the milk used in step (i) is defatted.

15. The method of claim 14, wherein the milk used in step (i) is either raw or pasteurized.

16. The method of claim 15, wherein the method further comprises subjecting the extracellular vesicles obtained from step (ii) to a filtration that removes extracellular vesicles with particle sizes less than 20 nm and a separate filtration that removes vesicles with particle sizes greater than 600 nm.

17. A composition comprising milk extracellular vesicles obtained from the method of claim 16.

18. The composition of claim 17 wherein the composition is lyophilized by either flash freezing or controlled freeze, followed by primary drying, secondary drying, and vacuum capping of samples.

19. A composition comprising Lyophilized milk extracellular vesicles obtained from the composition of claim 18, supplemented with 100 μM Tryptophan and 50 mM Trehalose
